(19) United States
(12) Patent Application Publication
(10) Pub. No.: US 2023/0165831 A1
(43) Pub. Date: Jun. 1, 2023

(54) METHOD AND COMPOSITION FOR RENDERING CANCER CELLS SUSCEPTIBLE TO TREATMENT BY TARGETED ONCOGENETIC DRIVERS

(71) Applicant: IMPACT BIOLIFE SCIENCE, INC., HOUSTON, TX (US)

(72) Inventor: Daryl L. THOMPSON, Winter Haven, FL (US)

(21) Appl. No.: 17/530,956

(22) Filed: Nov. 19, 2021

(51) Int. Cl. A61K 31/353; A61K 31/337; A61K 31/704; A61K 31/4745; A61K 33/243; A61P 35/00

(52) U.S. Cl. CPC A61K 31/353; A61K 31/337; A61K 31/704; A61K 31/4745; A61K 33/243; A61P 35/00

(57) ABSTRACT

Method and composition for treating or limiting the occurrence of cancer includes combining a polyphenol with a chemotherapeutic agent. Advantageously, the polyphenol may be myricetin including halogenated myricetin such as mono- or dichlorinated myricetin and the chemotherapeutic agent includes but is not limited to paclitaxel, doxorubicin, topotecan and cisplatin. The polyphenol such as myricetin including halogenated such as chlorinated myricetin can be combined in a single pharmaceutical composition with chemotherapeutic agent or a method can be coadministering separate compositions of a polyphenol and a therapeutic agent to treat or limit the occurrence of cancer.

### FIELD OF THE INVENTION

The present invention relates to a method and treatment of cancer and in particular a method and treatment for limiting the occurrence of cancer by enhancing chemotherapeutic agents.

### BACKGROUND OF THE INVENTION

Cancer is characterized by the uncontrolled proliferation of abnormal cells within the body. Some conventional therapeutic treatments of cancer include anticancer therapeutic agents which target the abnormal cells. The anti-chemotherapeutic agents target the abnormal cells and advantageously lead to their epitasis or cell death. Proviral Integration site for Maloney murine leukemia virus (MuLV) kinase (hereinafter PIM kinase) expression is inversely correlated to patient survival when undergoing cancer therapy. Natural inhibition of PIM kinase has been seen as an attractive target when treating cancer. However, a large number of small molecule PIM kinase inhibitors have been developed and have demonstrated promising results but with significant issues limiting PIM inhibitor effectiveness and having poor viability due to a limited target sensitivity.

### SUMMARY OF THE INVENTION

The present invention is directed to methods and compositions for treating or limiting the occurrence of cancer. The composition includes a combination of one or more conventional anticancer chemotherapy agents with one or more polyphenols. Advantageously, the polyphenol enhances bioavailability of the anticancer therapeutic agents. The polyphenol can be but is not limited to myricetin and could be other polyphenols such as chrysin. The polyphenol such as myricetin can be in its native form or can be halogenated and includes monochlorinated myricetin and dichlorinated myricetin. These three forms of myricetin have formulas I-III as follows:

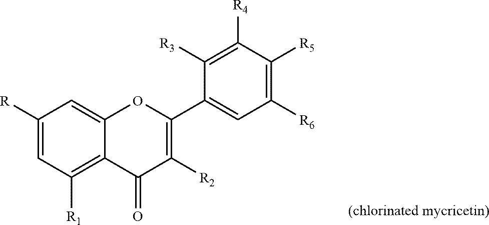

wherein R, R₁, R₂, R₄, R₅, and R₆ are a hydroxyl group or chlorine, R₃ is hydrogen; and
wherein, at least one of R, R₁, R₂, R₄, R₅, and R₆ is chlorine.

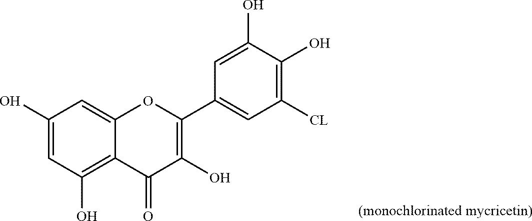

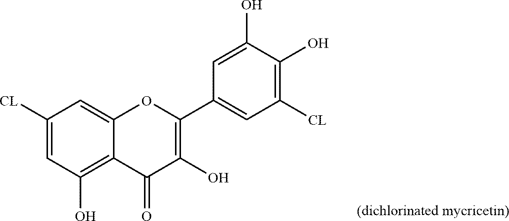

Conventional anticancer chemotherapeutic agents for combination with the polyphenol include but are not limited to those listed in Table 1 below:

TABLE 1

| | |
|---|---|
| Generic name of chemotherapeutic agent | Tradename / brand name |
| paclitaxel | Taxol |
| doxorubicin | Adriamycin, Lipodox, Lipodox 50, and Doxil |
| topotecan | Hycamtin |
| cisplatin (AKA: cisplatinum, platamin, neoplatin, cismaplat, and cis-diamminedichloroplatinum(II) (CDDP)) | Platinol |

The polyphenol and chemotherapeutic agent can be combined together in a single pharmaceutical composition. Alternatively, instead of a single pharmaceutical composition which combines the polyphenol with the chemotherapeutic agent, separate compositions, i.e., a polyphenol composition and an anticancer chemotherapeutic agent can be separately administered to a patient in need of treatment therefrom in order to treat or prevent the occurrence of cancer.

Further, the present method and composition advantageously increase the effectiveness of anticancer therapeutics by inhibiting a primary driver of cancer replication and metastasis, namely the overexpression of the PIM oncogene (proviral integration site for Maloney murine leukemia virus) and its downrange cancer promoting mechanism known as oncogenesis.

Further, the present method and composition advantageously increases the effectiveness of anticancer therapeutics by increasing bioviability due to inhibition of CYP450 isozymes.

Further in one advantageous form, the method and composition results in an increase in effectiveness of anticancer therapeutics including those of radiometric or radioactive nature by protecting unaffected cells while increasing sensitivity of cancerous cells and tumors. For example, the method and composition can increase the effectiveness of anticancer compounds by protecting uncancerous or healthy cells from chemo-assault (chemo-protective) while increasing the sensitivity of cancerous cells to chemotherapy.

It will be appreciated that based on a mechanism of how the polyphenol including myricetin including halogenated such as chlorinated myricetin effects cancer cells and in view of the polyphenol’s combination with anticancer chemotherapeutic agents disclosed herein functions, that additional therapeutic methods for treating cancer and limiting the occurrence of cancer include combining a polyphenol including myricetin such as halogenated including chlorinated myricetin include, but are not limited to, combining the polyphenol with one or more of immunotherapy, stem cell therapy, radiation, and hormone treatment.

The present invention, in one form thereof, is directed to a pharmaceutical composition comprising myricetin and an anticancer chemotherapeutic agent. The anticancer chemotherapeutic agent can be selected from the group consisting of paclitaxel, doxorubicin, topotecan and cisplatin. The myricetin can be native or unmodified myricetin or a halogenated form including chlorinated myricetin.

Further, the chlorinated myricetin can have a chemical formula I-III as follows:

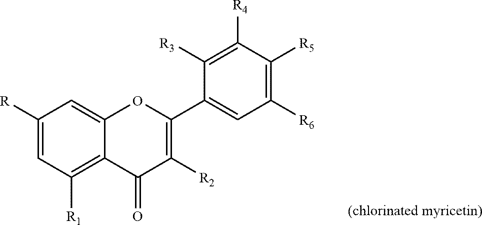

wherein R, R₁, R₂, R₄, R₅, and R₆ are a hydroxyl group or chlorine, R₃ is hydrogen; and
wherein, at least one of R, R₁, R₂, R₄, R₅, and R₆ is chlorine.

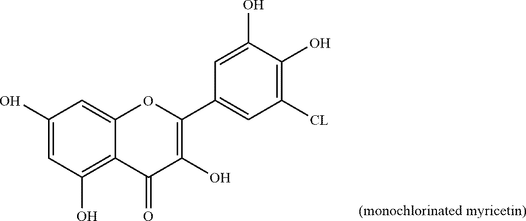

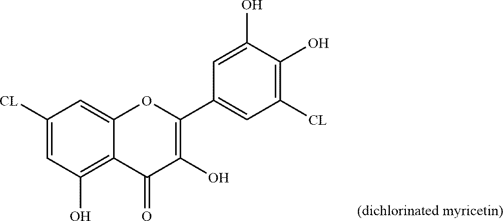

In one further alternative form, the pharmaceutical composition can treat or limit the occurrences of the following cancers: breast cancer, glioblastoma, prostate adenocarcinoma, kidney cancer, gastric cancer, colorectal cancer, liver cancer, pancreatic cancer, ovarian cancer, and lung adenocarcinoma.

The present invention, in another form thereof is directed to a method of treating or limiting the occurrence of cancer by co-administering a therapeutically effective amount of myricetin and a chemotherapeutic agent. The myricetin can be a modified myricetin or chlorinated myricetin including halogenated myricetin of formula I or mono-or dichlorinated myricetin of formulas II and III as follows:

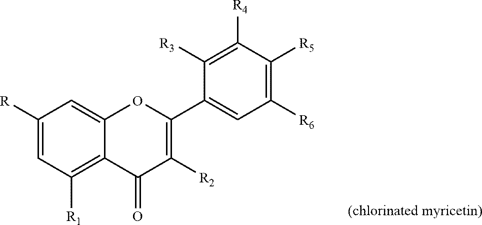

wherein R, R₁, R₂, R₄, R₅, and R₆ are a hydroxyl group or chlorine, R₃ is hydrogen; and
wherein, at least one of R, R₁, R₂, R₄, R₅, and R₆ is chlorine.

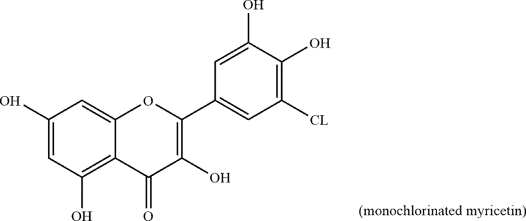

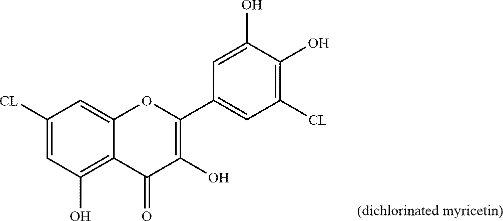

Advantageously, the anticancer chemotherapeutic agent is selected from the group consisting of paclitaxel, doxorubicin, topotecan and cisplatin.

The cancer treated includes but is not limited to those of breast cancer, glioblastoma, prostate adenocarcinoma, kidney cancer, gastric cancer, colorectal cancer, liver cancer, pancreatic cancer, ovarian cancer, and lung adenocarcinoma.

The present invention in another form thereof is directed to a method of downregulating PIM kinase comprising co-administering a therapeutically effective amount of myricetin and an anticancer chemotherapeutic agent to a patient in need thereof. The PIM kinase includes PIM-1, PIM-2 and PIM-3.

Advantageously, downregulating PIM kinase results in the upregulation or stimulative expression tumor suppressor mechanisms including retinoblastoma protein (pRb), cyclin dependent kinase inhibitor 2A, CDKN2A multiple tumor suppressor 1 (p16), ARF tumor suppressor (p14arf), transforming growth factor (TGF-beta), adenomatous polyposis coli (APC), breast cancer type 1 susceptibility protein (BRCA1 ), and / or tumor protein / cellular tumor antigen (p53).

Further, downregulating PIM kinase results in the upregulation or simulative expression of metastasis suppressor proteins including breast cancer metastasis suppressor 1 (BRMS1), mediator of RNA polymerase II transcription subunit 23 (CRSP3), developmentally regulated GTP binding protein1 (DRG1), cluster/differentiation 82 (CD82), serum deprived response protein (SDPR), kisseptin 54 (KISS1), nucleoside diphosphate kinase A (NME1), tissue inhibitor of metalloproteinase (TIMPs), or dual specificity mitogen activated protein kinase 4 (MKK4).

In addition, advantageously downregulating PIM kinase results in downrange attenuation of pro-inflammatory regulators including tumor necrosis factor alpha (TNF-α), nuclear factor kappa light chain enhancer of activated B cells (NF-kB), or lymphotoxin beta receptor (LTBR).

Further, advantageously downregulating PIM kinase results in inhibition of downrange pro-oncogenetic driving mechanisms and gene expression including such as chromobox protein homolog 3 (CBX3), m-phase inducer phosphatase 1 (CDC24A), heat shock protein 90kDa alpha-member A1, nuclear factor / activated T-cells-cytoplasmic 1 (NFATC1), nuclear mitotic apparatus protein 1, cyclin dependent kinase inhibitor 1 (P21), staphylococcal nuclease domain containing protein 1 (SND1), transcription factor p65 (RELA), dual-specificity phosphatase (Cdc25), P13K kinase, hypoxia induced factor 1 alpha (HIF1A), MYC, Janus kinase signal transducer and activator of transcription proteins (JAK-STAT), mammalian target of rapamycin (mTOR), FK506 binding protein 12 rapamycin associated protein 1 (FRAP1), protein kinase B, basic fibroblast growth factor (FGF2), vascular endothelial growth factor (VEGF), proto-oncogene (RET), Ras GTPase, RAF proto-oncogene serine/therine protein kinase (c-Raf), cyclin dependent kinase (CDK), tyrosine-protein kinase (SYK), platelet derived growth factor (PDGF).

Combining a polyphenol such as myricetin including halogenated myricetin such as chlorinated myricetin with conventional chemotherapeutic treatments which include but are not limited to chemotherapeutic agents paclitaxel, doxorubicin, topotecan and cisplatin has a synergistic effect over the aforementioned conventional chemotherapeutic treatments.

### DETAILED DESCRIPTION

Pharmaceutical compositions and methods are directed to PIM expression in cancerous cell lines. PIM is directly responsible for driving cancer genesis by four (4) key mechanisms, (1) stimulating onco-genetic drivers, and (2) pro-inflammatory markers while suppressing (3) tumor suppressors and (4) metastasis suppressors. Inhibition of PIM by a polyphenol or and electrophilically enhanced (halogenated) polyphenol reduces a cancerous cells survivability and increases the effectiveness of anticancer therapeutic regimes reversing PIM activated and suppressed mechanisms. Inhibition of PIM driven onco-genetic drivers essentially causes a powerful downstream attenuation of key pro-cancer mechanisms such as CBX3, CDC24A, 90kDa, NFATC1, P21, SND1, RELA, Cdc25, P13K, HIF1A, Myc, JAK-STAT, mTOR, FRAP1, FGF2, VEGF, RET, RAF, c-RAF, CDK, SYK, PDGF. Inhibition of PIM proinflammatory markers allows for a significant reduction in the stimulation of onco-genetic drivers as well as PIM itself essentially terminating and slowing a redundant feedback mechanism that enables cancer to increase. This is also a primary driver for cancer cell resistance.

The specific pro-inflammatory drivers affected by PIM inhibition are: TNF-α, NF-kB, and LTBR. Inhibition of PIM and the subsequent reduction and downregulation of the above two mechanisms further improve the efficacy of anti-cancer therapy by allowing for the activation and stimulation of tumor suppressor activity such as pRb, CDKN2A, p16, p14arf, TGF-β, APC, BRCA1 and P53. Inhibition of PIM, the onco-genetic drivers, pro-inflammatory mechanisms coupled with the expression of tumor suppressors work synergistically to stimulate expression of metastasis suppressor proteins. This works further to weaken cancer cell resistance to treatment as well as significantly increase the therapeutic value of anticancer treatment paradigms.

The primary metastasis genes expressed are BRMS1, CRSP3, DRG1, CD82, SDPR, KISS1, NME1, TIMPs, and MKK4. PIM inhibitors are combined with myricetin (a polyphenol) and a halogenated polyphenol such as those of formulas I-III with the anti-cancer therapeutics paclitaxel, doxorubicin, topotecan and cisplatin. These combinations were assayed against several cancer cell lines with multiple replications and concentrations. These studies resulted in significant increases in efficacy with all drugs, all, combinations, against all cancer cell lines.

### Effective Amount

The preferred dose for administration of a polyphenol including myricetin (native or halogenated) with a chemotherapeutic agent, including but not limited to paclitaxel, doxorubicin, topotecan, and cisplatin, in accordance with the present invention is that amount which will be effective in treating or limiting the occurrence of cancer, breast cancer, glioblastoma, prostate adenocarcinoma, kidney cancer, gastric cancer, colorectal cancer, liver cancer, pancreatic cancer, ovarian cancer, and lung adenocarcinoma, by inhibition of the (1) CYP450 enzyme isozyme superfamily and/or (2) the proviral integration site for a Moloney murine leukemia virus (PIM) kinase (oncogenic) expression, and one would readily recognize that this amount will vary greatly depending on the nature and extent of the disease and the condition of a patient. An “effective amount” of the pharmaceutical composition to be used in accordance with the invention is intended to mean a nontoxic but sufficient amount of the agent, such that the desired prophylactic or therapeutic effect is produced. Thus, the exact amount of the pharmaceutical composition that is required will vary from subject to subject, depending on the species, age, and general condition of the subject, the severity of the condition being treated, the particular carrier or adjuvant being used and its mode of administration, and the like. Similarly, the dosing regimen should also be adjusted to suit the individual to whom the composition is administered and will once again vary with age, weight, metabolism, etc. of the individual. Accordingly, the “effective amount” of any particular pharmaceutical composition will vary based on the particular circumstances, and an appropriate effective amount may be determined in each case of application by one of ordinary skill in the art using only routine experimentation.

### Experiments

The present method and pharmaceutical composition will now be described with regard to experiments demonstrating efficacy.

The experimental study tested the synergistic effects of eight (8) combinations between six (6) drugs; monochlorinated myricetin (formula II), myricetin, paclitaxel, doxorubicin, topotecan and cisplatin on ten (10) cancer cell lines (U87MG, T47D, A549, SK-OV-3, MKN45, SK-Hep-1, Capan-1, HT29, 786-O, LNCaP). The results of eighty (80) viability assays demonstrated for each cell line there are synergistic effects.

For breast cancer line T47D cells, combinations of 100 µM LB-1 and 2000 nM doxorubicin, 100 µM LB-1 and 2000 nM topotecan, 100 µM LB-1 and 30 µM cisplatin, caused dramatical growth inhibition, with the growth inhibition rate being 86.95%, 97.18%, 82.48%, respectively, while combinations of 100 µM myricetin and 2000 nM doxorubicin, 100 uM myricetin and 10000 nM topotecan, 100 µM LB-1 and 200 nM paclitaxel caused mediate growth inhibition with the growth inhibition rate being 77.64%, 67.56%, 75.50%, respectively.

For glioblastoma line U87MG cells, combinations of 50 µM LB-1 and 400 nM doxorubicin, 50 µM LB-1 and 2000 nM topotecan, 50 µM LB-1 and 30 µM cisplatin, 0.16 µM myricetin and 400 nM doxorubicin, 100 µM myricetin and 30 µM cisplatin generated dramatically significant growth inhibition, with the growth inhibition rate being 99.03%, 98.84%, 95.97%, 93.15%, 90.10%, respectively. Combinations of 100 µM myricetin and 10000 nM topotecan, 50 µM LB-1 and 1000 nM taxol, 100 µM myricetin and 200 nM taxol, also caused quite high growth inhibition rate (87.63%, 83.65%, 77.49%), showing that U87MG cells have a very good response for the tested combination treatment.

For prostate adenocarcinoma line LNCap cells, combinations of 50 µM LB-1 and 1000 nM taxol, 50 µM LB-1 and 400 nM doxorubicin, 50 µM LB-1 and 2000 nM topotecan, 50 µM LB-1 and 30 µM cisplatin, 4 µM myricetin and 2000 nM doxorubicin, 100 µM myricetin and 10000 nM topotecan, all have quite significantly high growth inhibition rates (98.57%, 99.78%, 99.82%, 99.48%, 97.73%, 91.49%, respectively), while combinations of 0.8 µM myricetin and 40 nM taxol, 100 µM myricetin and 30 µM cisplatin have a moderate inhibition rate (65.76%, 77.31%, respectively).

For kidney cancer line 786-O cells, combinations of 50 µM LB-1 and 1000 nM taxol, 50 µM LB-1 and 400 nM doxorubicin, 20 µM LB-1 and 2000 nM topotecan, 50 µM LB-1 and 6 µM cisplatin, 100 µM myricetin and 1000 nM taxol, 100 µM myricetin and 10000 nM doxorubicin, 100 µM myricetin and 10000 nM topotecan, 100 µM myricetin and 30 µM cisplatin all caused more than 80% growth inhibition. Combinations of 20 µM LB-1 and 2000 nM topotecan, 50 µM LB-1 and 6 µM cisplatin, 100 µM myricetin and 10000 nM topotecan have more than 95% growth inhibition rate.

For gastric cancer line MKN45 cells, combinations of 50 µM LB-1 and 200 nM taxol, 50 µM LB-1 and 2000 nM doxorubicin, 50 µM LB-1 and 2000 nM topotecan, 50 µM LB-1 and 30 µM cisplatin, 20 µM myricetin and 2000 nM doxorubicin, 100 µM myricetin and 2000 nM topotecan, 100 µM myricetin and 30 µM cisplatin, showed significant growth inhibition of more than 90%, while only one combination of 20 µM myricetin and 1000 nM taxol had a growth inhibition rate of 72.77%.

For colorectal cancer line HT29 cells, all combinations of 100 µM LB-1 and 1000 nM taxol, 20 µM LB-1 and 2000 nM doxorubicin, 100 µM LB-1 and 2000 nM topotecan, 100 µM LB-1 and 30 µM cisplatin, 100 µM myricetin and 8 nM taxol, 4 µM myricetin and 2000 nM doxorubicin, 100 µM myricetin and 2000 nM topotecan, 100 µM myricetin and 30 µM cisplatin showed a greatly significant growth inhibition rate (87.34%, 95.77%, 98.29%, 94.01%, 97.60%, 93.72%, 95.95%, respectively).

Liver cancer line SK-HEP-1 cells also had a dramatically significant growth inhibition for all combinations, with combinations of 20 µM LB-1 and 1000 nM taxol, 100 µM LB-1 and 10000 nM doxorubicin, 100 uM LB-1 and 10000 nM topotecan, 100 µM LB-1 and 30 µM cisplatin, 100 uM myricetin and 1000 nM taxol, 100 uM myricetin and 1000 nM taxol/doxorubicin, 100 uM myricetin and 10000 nM topotecan, 100 µM myricetin and 30 µM cisplatin, causing growth inhibition rates of 89.75%, 99.85%, 99.81%, 95.36%, 99.67%, 99.98%, 99.95%, 99.77%, respectively.

For pancreatic cancer line Capan-1 cells, combinations of 50 µM LB-1 and 2000 nM doxorubicin, 50 uM LB-1 and 2000 nM topotecan, 50 µM LB-1 and 30 µM cisplatin, 100 uM myricetin and 2000 nM topotecan caused significant growth inhibition rates (90.49%, 93.49%, 89.95%, 92.11 %, respectively). Combinations of 50 µM LB-1 and 1000 nM taxol, 100 uM myricetin and 1000 nM taxol, 4 µM myricetin and 2000 nM taxol/doxorubicin, 100 µM myricetin and 30 µM cisplatin also caused high growth inhibition rates of 78.30%, 72.31 %, 87.27%, 86.28%, respectively.

For ovarian cancer line SK-OV3 cells, all combinations of 50 µM LB-1 and 1000 nM taxol, 50 µM LB-1 and 2000 nM doxorubicin, 50 uM LB-1 and 10000 nM topotecan, 20 µM LB-1 and 30 µM cisplatin, 100 uM myricetin and 1000 nM Taxol, 100 µM myricetin and 10000 nM taxol/doxorubicin, 100 uM myricetin and 10000 nM topotecan, 100 µM myricetin and 30 µM cisplatin showed dramatically significant growth inhibition rates (88.31 %, 95.51 %, 96.23%, 93.31 %, 92.69%, 98.68%, 96.35%, 97.69%, respectively). These data showed that SK-OV3 cells are responsive quite well to all combinations.

For lung adenocarcinoma line A549 cells, combinations of 20 µM LB-1 and 2000 nM doxorubicin, 50 uM LB-1 and 10000 nM topotecan, 50 µM LB-1 and 30 µM cisplatin, 100 µM myricetin and 10000 nM taxol/doxorubicin, 100 uM myricetin and 10000 nM topotecan, 100 µM myricetin and 30 µM cisplatin, all had a more than 90% growth inhibition rates (97.56%, 99.39%, 95.00%, 96.94%, 96.32%, 89.71%, respectively). Combinations of 50 µM LB-1 and 200 nM taxol, 20 uM myricetin and 1000 nM taxol also had moderate to high growth inhibition rates (80.95%, 79.36%, respectively).

### Objectives

To measure the cell inhibition effect of compounds and synergy effect of test articles on ten (10) cell lines using standard CTG assay.

### Study Design

For measuring synergy effect, the cell line will be treated with monochlorinated myricetin (formula II) or myricetin combined with taxol, doxorubicin, topotecan, or cis-platinum in 6×6 combo matrix for 72 h at a temperature of 37° C. with 5% CO₂ and 95% humidity.

### Materials and Methods

### 1. Compounds Information

TABLE 2

| Compound | MW | Master stock (mM) | Compound needed to make master stock (mg) | Volume of master stock (µL) |
|---|---|---|---|---|
| monochlorinated myricetin (formula II) | 336.68 | 25 | 8.77 | 1042 |
| myricetin | 318.24 | 100 | 15.9 | 500 |
| taxol | 853.91 | 10 | 4.3 | 500 |
| doxorubicin | 579.98 | 10 | 2.9 | 500 |
| topotecan | 421.45 | 10 | 2.1 | 500 |
| cis-platinum | 300.05 | 3.333 | 1 | 1000 |

### 2. Preparation of Compounds Solution

Solid compound of monochlorinated myricetin (formula II), myricetin, taxol, doxorubicin and topotecan were solubilized in DMSO to a stock solution of 25 mM, 100 mM or 10 mM. Solid compound of cis-platinum was solubilized in PBS to a stock solution of 3.333 mM.

Note: Compound Addition was performed according to the plate maps.

### 3. Cell Lines

TABLE 3

| Cell Line | | Culture Medium |
|---|---|---|
| 1 | U87MG | MEM+10%PBS+1%NEAA+1%NAP+1% P.S. |
| 2 | T47D | 1640+10%PBS+1% P.S. |
| 3 | A549 | F-12K Nutrient Mixture+10%PBS+1% P.S. |
| 4 | SK-OV-3 | 1640+10%PBS+1% P.S. |
| 5 | MKN45 | 1640+20%PBS+1% P.S. |
| 6 | SK-Hep-1 | MEM+10%PBS+1%NEAA+1% P.S. |
| 7 | Capan-1 | IMDM+20%PBS+1% P.S. |
| 8 | HT-29 | McCoy’s 5A+10%PBS+1% P.S. |
| 9 | 786-O | 1640+10%PBS+1% P.S. |
| 10 | LNCaP | 1640+10%PBS+1% P.S. |

### 4. Reagents

CellTiter-Glo® Luminescent Cell Viability Assay (Promega-Cat# G7573). Stored at -20° C.

IMDM Medium (Gibco Cat# 12440-053). Stored at 4° C.

MEM Medium (Gibco Cat# 11095-080). Stored at 4° C.

McCoy’s 5A Medium (Gibco Cat# 16600-082). Stored at 4° C.

RPMI 1640 Medium (Gibco Cat# 11415-064). Stored at 4° C.

F-12K Nutrient Mixture Medium (Gibco Cat# 21127-022). Stored at 4° C.

Trypsin-EDTA (0.25%) (STEMCELL-Cat# 09701). Stored at -20° C.

FBS (ExCell Bio-Cat# FND500). Stored at -20° C.

Phosphate Buffered Saline (PBS) (Gibco-REF#C20012500BT). Stored at RT (20° C.).

Penicillin/Streptomycin (100x) (Gibico-REF#15140-122). Stored at 4° C.

MEM NEAA (100x) (Gibico Cat# 11140-050 100 mL). Stored at 4° C.

Sodium Pyruvate (100 mM) (Gibico Cat# 11360-070 100 mL). Stored at 4° C.

Dimethyl sulfoxide (DMSO) 100 ML (Sigma-Cat# D2650-100ML). Stored at RT (20° C.), under inert conditions.

### 5. Instruments

Cell counter: Counter star (Ruiyu-biotech)
CO₂ cell incubator: MCO-15AC (Thermo Fisher)
Pipette: BioHit Multichannel, 50-1200 µL (RAININ Multichannel).
Pipette: 0.2-10 µL, 10-300 µL, 5-50 µl (Eppendorf)
Centrifuge: Centrifuge ST 40R (Thermo Fisher)
Water system: Milli-Q Reference system (Millipore)
Perkin Elmer Envision 2104 Multilabel Reader (No. 01-094-0002)

### 6. Assay Protocol

### 6.1 Preparation of Cell Assay Plates: Day 1

1) Pre-warm Trypsin-EDTA (0.25%), cell medium with 37° C. water bath.

2) Observe cells under microscope to assess the degree of confluency and confirm the absence of bacterial and fungal contaminants.

3) Remove medium, wash cells with 10 mL PBS twice. Add 2 mL 0.25% Trypsin/EDTA reagent for a T-75 flask. Put flask in the incubator for a few minutes, or until cells have detached. Add 7 mL of fresh cell medium contain 10% FBS, rinse the cells and transfer to a centrifuge tube.

4) Centrifuge the collected cells at 200 g for 5 minutes, at room temperature.

5) After centrifugation, discard the supernatant. Resuspend the cell pellet with 5 mL complete cell medium.

6) Remove 20 µL of the resuspended cells to count cells. Count cells by adding 20 µL cell suspension to 20 µL dye with Cell Counter Star, record live cell number and viability in cell tracking sheet.

7) Using complete cell medium, adjust the volume of the suspension to achieve a cell concentration.

a. U87MG cell lines, for compounds and vehicle groups, the seeding density of cell lines is 2000 cells/80 µL per well. For blank groups, add 100 µL medium. For PBS groups, add 100 µL PBS.

b. A549, T47D, SK-OV-3, MKN45, SK-Hep-1, HT-29, Capan-1, 786-O and LNCaP cell line, for compounds and vehicle groups, the seeding density of cell lines is 3000 cells/80 µL per well. For blank groups, add 100 µL medium. For PBS groups, add 100 µL PBS.

8) Incubate the U87MG, A549, T47D, SK-OV-3, MKN45, SK-Hep-1, HT-29, Capan-1, 786-O and LNCaP cells overnight at 37° C./5% CO₂.

### 6.2 Compounds Dosage Gradient Solution Preparation: Day 2

1) Solid compound of monochlorinated myricetin (formula II), myricetin, taxol, doxorubicin, topotecan was solubilized in DMSO to a stock solution of 25 mM, 100 mM or 10 mM. Solid compound of cis-platinum was solubilized in PBS to a stock solution of 3.333 mM.

2) Dilute the compounds according to the Table 4, Table 5, Table 6, Table 7, Table 8 and Table 9.

TABLE 4

Taxol Diluted Methods

| compounds | compounds concentration (mM) | Added compounds volume(µL) | Added DMSO volume(µL) | concentration after dilution(mM) | Added compounds volume(µL) | added Medium volume(µL) | concentration after dilution(µM) | added compounds volume(µL) | cell suspension volume(µL) | compounds final concentration (µM) |
|---|---|---|---|---|---|---|---|---|---|---|
| taxol | 10 | 50 | 450 | 1 | 10 | 990 | 10.000 | 10 | 90 | 1.00000 |
| | 1 | 100 | 400 | 0.20000 | 10 | 990 | 2.000 | 10 | 90 | 0.200 |
| | 0.2 | 100 | 400 | 0.04000 | 10 | 990 | 0.400 | 10 | 90 | 0.040 |
| | 0.04 | 100 | 400 | 0.00800 | 10 | 990 | 0.080 | 10 | 90 | 0.008 |
| | 0.008 | 100 | 400 | 0.00160 | 10 | 990 | 0.016 | 10 | 90 | 0.0016 |

TABLE 5

Topotecan Diluted Methods

| compounds | compounds concentration (mM) | added compounds volume(µL) | added DMSO volume(µL) | concentration after dilution(mM) | added compounds volume(µL) | added Medium volume(µL) | concentration after dilution(µM) | added compounds volume(µL) | cell suspension volume(µL) | compounds final concentration (µM) |
|---|---|---|---|---|---|---|---|---|---|---|
| topotecan | 10 | / | / | 10 | 10 | 990 | 100.000 | 10 | 90 | 10.00000 |
| | 10 | 100 | 400 | 2.00000 | 10 | 990 | 20.000 | 10 | 90 | 2.000 |
| | 2.0 | 100 | 400 | 0.40000 | 10 | 990 | 4.000 | 10 | 90 | 0.400 |
| | 0.40 | 100 | 400 | 0.08000 | 10 | 990 | 0.800 | 10 | 90 | 0.080 |
| | 0.080 | 100 | 400 | 0.01600 | 10 | 990 | 0.160 | 10 | 90 | 0.016 |

TABLE 6

Doxorubicin Diluted Methods

| compounds | compounds concentration (mM) | added compounds volume(µL) | added DMSO volume(µL) | concentration after dilution(mM) | added compounds volume(µL) | added Medium volume(µL) | concentration after dilution(µM) | added compounds volume(µL) | cell suspension volume(µL) | compounds final concentration (µM) |
|---|---|---|---|---|---|---|---|---|---|---|
| doxorubicin | 10 | / | / | 10 | 10 | 990 | 100.000 | 10 | 90 | 10.00000 |
| | 10 | 100 | 400 | 2.00000 | 10 | 990 | 20.000 | 10 | 90 | 2.000 |
| | 2.0 | 100 | 400 | 0.40000 | 10 | 990 | 4.000 | 10 | 90 | 0.400 |
| | 0.40 | 100 | 400 | 0.08000 | 10 | 990 | 0.800 | 10 | 90 | 0.080 |
| | 0.080 | 100 | 400 | 0.01600 | 10 | 990 | 0.160 | 10 | 90 | 0.016 |

TABLE 7

Doxorubicin Diluted Methods

| compounds | compounds concentration (mM) | added compounds volume(µL) | added PBS volume(µL) | concentration after dilution(mM) | added compounds volume(µL) | added Medium volume(µL) | concentration after dilution(µM) | added compounds volume(µL) | cell suspension volume(µL) | compounds final concentration (µM) |
|---|---|---|---|---|---|---|---|---|---|---|
| cisplatin | 3.333 | / | / | 3.333 | 75 | 758.25 | 300.000 | 10 | 90 | 30.00000 |
| | 3.333 | 100 | 400 | 0.66660 | 75 | 758.25 | 60.000 | 10 | 90 | 6.000 |
| | 0.7 | 100 | 400 | 0.13332 | 75 | 758.25 | 12.000 | 10 | 90 | 1.200 |
| | 0.13 | 100 | 400 | 0.02666 | 75 | 758.25 | 2.400 | 10 | 90 | 0.240 |
| | 0.027 | 100 | 400 | 0.00533 | 75 | 758.25 | 0.480 | 10 | 90 | 0.048 |

TABLE 8

Myricetin Diluted Methods

| compounds | compounds concentration (mM) | added compounds volume(µL) | added DMSO volume(µL) | concentration after dilution(mM) | added compounds volume(µL) | added Medium volume(µL) | concentration after dilution(µM) | added compounds volume(µL) | cell suspension volume(µL) | compounds final concentration (µM) |
|---|---|---|---|---|---|---|---|---|---|---|
| myricetin | 100 | / | / | 100 | 13 | 1287 | 1000.000 | 10 | 90 | 100.00000 |
| | 100 | 100 | 400 | 20.00000 | 13 | 1287 | 200.000 | 10 | 90 | 20.000 |
| | 20.0 | 100 | 400 | 4.00000 | 13 | 1287 | 40.000 | 10 | 90 | 4.000 |
| | 4.00 | 100 | 400 | 0.80000 | 13 | 1287 | 8.000 | 10 | 90 | 0.800 |
| | 0.800 | 100 | 400 | 0.16000 | 13 | 1287 | 1.600 | 10 | 90 | 0.160 |

TABLE 9

Monochlorinated Myricetin (Formula II) Diluted Methods

| compounds | compounds concentration (mM) | added compounds volume(µL) | added DMSO volume(µL) | concentration after dilution (mM) | added compound s volume (µL) | added Medium volume(µL) | concentration after dilution (µM) | added compounds volume(µL) | cell suspension volume (µL) | compounds final concentrati on (µM) |
|---|---|---|---|---|---|---|---|---|---|---|
| monochlori nated myricetin (formula II) | 25 | 100 | 100 | 12.5 | 48 | 1152 | 500.000 | 10 | 90 | 50.00000 |
| | 12.5 | 200 | 300 | 5 | 48 | 1152 | 200.000 | 10 | 90 | 20.000 |
| | 5 | 100 | 400 | 1 | 48 | 1152 | 40.000 | 10 | 90 | 4.000 |
| | 1 | 100 | 400 | 0.2 | 48 | 1152 | 8.000 | 10 | 90 | 0.800 |
| | 0.2 | 100 | 400 | 0.04 | 48 | 1152 | 1.600 | 10 | 90 | 0.160 |

3) Dilute the vehicle according to the Table 10 - Table 12

TABLE 10

Vehicle for Monochlorinated Myricetin (Formula II) Diluted Methods

| vehicle | compound initial concentration(%) | Dilution(added compound(uL)) | Dilution(added medium(uL)) | Diluted concentration(%) | Addeded compound volume(uL) | Volume of liquid in the well(uL) | compound final concentration(%) |
|---|---|---|---|---|---|---|---|
| DMSO | 100% | 500 | 12000 | 4% | 10 | 90 | 0.4% |

TABLE 11

Vehicle for Myricetin, Taxol, Doxorubicin, Topotecan Diluted Methods

| vehicle | compound initial concentration(%) | Dilution(added compound(uL)) | Dilution(added medium(uL)) | Diluted concentration(%) | Addeded compound volume(uL) | Volume of liquid in the well(uL) | compound final concentration(%) |
|---|---|---|---|---|---|---|---|
| DMSO | 100% | 200 | 19800 | 1% | 10 | 90 | 0.1% |

TABLE 12

Vehicle for cisplatin Diluted Methods

| vehicle | compound initial concentration(%) | Dilution(added compound(uL)) | Dilution(added medium(uL)) | Diluted concentration(%) | Addeded compound volume(uL) | Volume of liquid in the well(uL) | compound final concentration(%) |
|---|---|---|---|---|---|---|---|
| PBS | 100% | 1000 | 10110 | 9% | 10 | 90 | 0.9% |

4) Add 10 µL/well of the 10X compounds work stock solutions (myricetin or monochlorinated myricetin (formula II)) and 10 µL/well of the 10X compounds work stock solutions (taxol or doxorubicin or topotecan or cisplatin) from step 2 and step 3 to 80 µL of cell culture in corresponding wells for 1X final concentrations as in plate map. Final volume is 100 µL per well at this point.

5) Incubate the plates at 37° C./5% CO₂ for 72 hours.

### 6.3 Detect: Day 5

1) Incubate the plates at room temperature and away from light for 30 minutes.

2) Thaw three vials of CellTiter-Glo® Reagent at room temperature and equilibrate them to room temperature prior to use. Avoid light.

3) Add 100 µL /well of CellTiter-Glo® Reagent in each well. Avoid light.

4) Mix contents for 2 minutes on an orbital shaker.

5) Incubate plate at room temperature for 10 minutes to stabilize the luminescent signal.

6) Read plates in Envision.

7 Data Analysis

$$\begin{matrix}\text{The Relatively Cell viability of vehicle control} \left(\%\right) = \\ \left(\begin{matrix}\left(\text{LumTest article - LumBlank control}\right) / \\ \left(\text{LumVehicle control - LumBlank control}\right)\end{matrix}\right) \times 100 \%\end{matrix}$$

$$\begin{matrix}\text{The Relatively Cell inhibition of vehicle control} \left(\text{\%}\right) = \\ \left(1 - \text{The Relatively Cell viability of vehicle control}\right) \times 100 \%\end{matrix}$$

### 8 Results Summary

The Relatively Cell inhibition of vehicle control (%) of T47D, U87MG, LNCaP, HT-29, MKN45, 786-O, SK-Hep-1, Capan-1, SK-OV-3 and A549 cell lines.

Summary for 10 cell lines:

TABLE 13

| Cell line | Combined added compounds concentration | Maximum inhibition rate (%) |
|---|---|---|
| T47D | 100 µM LB-1 and 200 nM taxol | 75.50 |
| | 100 µM LB-1 and 2000 nM doxorubicin | 86.95 |
| | 100 µM LB-1 and 2000 nM topotecan | 97.18 |
| | 100 µM LB-1 and 30 µM cisplatin | 82.48 |
| | 0.8 µM myricetin and 40 nM taxol | 50.52 |
| | 100 µM myricetin and 2000 nM doxorubicin | 77.64 |
| | 100 uM myricetin and 10000 nM topotecan | 67.56 |
| | 100 µM myricetin and 30 µM cisplatin | 53.89 |
| U87MG | 50 µM LB-1 and 1000 nM taxol | 83.65 |
| | 50 µM LB-1 and 400 nM doxorubicin | 99.03 |
| | 50 µM LB-1 and 2000 nM topotecan | 98.84 |
| | 50 µM LB-1 and 30 µM cisplatin | 95.97 |
| | 100 µM myricetin and 200 nM taxol | 77.49 |
| | 0.16 µM myricetin and 400 nM doxorubicin | 93.15 |
| | 100 µM myricetin and 10000 nM topotecan | 87.63 |
| | 100 µM myricetin and 30 µM cisplatin | 90.10 |
| LNCap | 50 µM LB-1 and 1000 nM taxol | 98.57 |
| | 50 µM LB-1 and 400 nM doxorubicin | 99.78 |
| | 50 µM LB-1 and 2000 nM topotecan | 99.82 |
| | 50 µM LB-1 and 30 µM cisplatin | 99.48 |
| | 0.8 µM myricetin and 40 nM taxol | 65.76 |
| | 4 µM myricetin and 2000 nM doxorubicin | 97.73 |
| | 100 µM myricetin and 10000 nM topotecan | 91.49 |
| | 100 µM myricetin and 30 µM cisplatin | 77.31 |
| 786-O | 50 µM LB-1 and 1000 nM taxol | 93.76 |
| | 50 µM LB-1 and 400 nM doxorubicin | 92.11 |
| | 20 µM LB-1 and 2000 nM topotecan | 98.31 |
| | 50 µM LB-1 and 6 µM cisplatin | 95.28 |
| | 100 µM myricetin and 1000 nM taxol | 82.02 |
| | 100 µM myricetin and 10000 nM doxorubicin | 85.17 |
| | 100 µM myricetin and 10000 nM topotecan | 96.42 |
| | 100 µM myricetin and 30 µM cisplatin | 93.88 |
| MKN-45 | 50 µM LB-1 and 200 nM taxol | 91.55 |
| | 50 µM LB-1 and 2000 nM doxorubicin | 96.04 |
| | 50 µM LB-1 and 2000 nM topotecan | 97.87 |
| | 50 µM LB-1 and 30 µM cisplatin | 94.06 |
| | 20 µM myricetin and 1000 nM taxol | 72.77 |
| | 20 µM myricetin and 2000 nM doxorubicin | 95.19 |
| | 100 µM myricetin and 2000 nM topotecan | 95.03 |
| | 100 µM myricetin and 30 µM cisplatin | 92.27 |
| HT29 | 100 µM LB-1 and 1000 nM taxol | 87.34 |
| | 20 µM LB-1 and 2000 nM doxorubicin | 95.77 |
| | 100 µM LB-1 and 2000 nM topotecan | 98.29 |
| | 100 µM LB-1 and 30 µM cisplatin | 94.01 |
| | 100 µM myricetin and 8 nM taxol | 97.60 |
| | 4 µM myricetin and 2000 nM doxorubicin | 93.72 |
| | 100 µM myricetin and 2000 nM topotecan | 95.95 |
| | 100 µM myricetin and 30 µM cisplatin | 95.37 |
| SK-Hep-1 | 20 µM LB-1 and 1000 nM taxol | 89.75 |
| | 100 µM LB-1 and 10000 nM doxorubicin | 99.85 |
| | 100 uM LB-1 and 10000 nM topotecan | 99.81 |
| | 100 µM LB-1 and 30 µM cisplatin. | 95.36 |
| | 100 uM myricetin and 1000 nM taxol | 99.67 |
| | 100 uM myricetin and 1000 nM taxol/doxorubicin | 99.98 |
| | 100 uM myricetin and 10000 nM topotecan | 99.95 |
| | 100 µM myricetin and 30 µM cisplatin | 99.77 |
| Capan-1 | 50 µM LB-1 and 1000 nM taxol | 78.30 |
| | 50 µM LB-1 and 2000 nM doxorubicin | 90.49 |
| | 50 uM LB-1 and 2000 nM topotecan | 93.49 |
| | 50 µM LB-1 and 30 µM cisplatin. | 89.95 |
| | 100 uM myricetin and 1000 nM taxol | 72.31 |
| | 4 µM myricetin and 2000 nM taxol/doxorubicin | 87.27 |
| | 100 uM myricetin and 2000 nM topotecan | 92.11 |
| | 100 µM myricetin and 30 µM cisplatin | 86.28 |
| SK-OV-3 | 50 µM LB-1 and 1000 nM taxol | 88.31 |
| | 50 µM LB-1 and 2000 nM doxorubicin | 95.51 |
| | 50 uM LB-1 and 10000 nM topotecan | 96.23 |
| | 20 µM LB-1 and 30 µM cisplatin. | 93.31 |
| | 100 uM myricetin and 1000 nM taxol | 92.69 |
| | 100 µM myricetin and 10000 nM taxol/doxorubicin | 98.68 |
| | 100 uM myricetin and 10000 nM topotecan | 96.35 |
| | 100 µM myricetin and 30 µM cisplatin | 97.69 |
| A549 | 50 µM LB-1 and 200 nM taxol | 80.95 |
| | 20 µM LB-1 and 2000 nM doxorubicin | 97.56 |
| | 50 uM LB-1 and 10000 nM topotecan | 99.39 |
| | 50 µM LB-1 and 30 µM cisplatin. | 95.00 |
| | 20 uM myricetin and 1000 nM taxol | 79.36 |
| | 100 µM myricetin and 10000 nM taxol/doxorubicin | 96.94 |
| | 100 uM myricetin and 10000 nM topotecan | 96.32 |
| | 100 µM myricetin and 30 µM cisplatin | 89.71 |

T47D

TABLE 14

monochlorinated myricetin (formula II)+taxol
monochlorinated myricetin (formula II)+taxol-Relatively Cell inhibition of vehicle control(%)

| Tax (nM) LB-1 (µM) | 100 | 20 | 4 | 0.8 | 0.16 | 0 |
|---|---|---|---|---|---|---|
| 1000 | 72.51 | 46.95 | 40.20 | 40.68 | 39.62 | 38.04 |
| 200 | 75.50 | 48.03 | 41.52 | 41.07 | 40.90 | 41.96 |
| 40 | 73.59 | 45.70 | 42.59 | 43.99 | 40.77 | 42.16 |
| 8 | 71.60 | 41.31 | 34.95 | 34.65 | 34.77 | 33.17 |
| 1.6 | 73.16 | 28.31 | 15.94 | 14.78 | 12.08 | 17.03 |
| 0 | 69.94 | 23.49 | 17.05 | -0.73 | 3.75 | 3.08 |

monochlorinated myricetin (formula II)+taxol-STEDV

| Tax (nM) LB-1 (µM) | 100 | 20 | 4 | 0.8 | 0.16 | 0 |
|---|---|---|---|---|---|---|
| 1000 | 2.86 | 3.12 | 2.18 | 1.45 | 1.41 | 1.64 |
| 200 | 3.88 | 2.57 | 0.36 | 0.74 | 1.83 | 1.02 |
| 40 | 5.76 | 0.53 | 1.04 | 2.63 | 0.85 | 1.60 |
| 8 | 3.47 | 2.39 | 1.05 | 0.78 | 2.72 | 3.04 |
| 1.6 | 1.83 | 4.98 | 3.11 | 4.87 | 3.51 | 0.93 |
| 0 | 7.06 | 3.91 | 35.54 | 2.45 | 3.59 | 4.61 |

The highest inhibition rate is 75.50%, at concentration of 100 µM LB-1 and 200 nM taxol.
The inhibition rate is 69.94% at 100 µM LB-1 only, and the inhibition rate is 41.96% at 200 nM taxol only.

TABLE 15

monochlorinated myricetin (formula II)+doxorubicin
monochlorinated myricetin (formula II)+doxorubicin-Relatively Cell inhibition of vehicle control(%)

| Dox (nM) LB-1 (µM) | 100 | 20 | 4 | 0.8 | 0.16 | 0 |
|---|---|---|---|---|---|---|
| 10000 | 62.97 | 45.89 | 45.16 | 44.60 | 44.68 | 45.28 |
| 2000 | 86.95 | 59.08 | 54.49 | 52.62 | 53.10 | 51.6 |
| 400 | 73.67 | 49.43 | 49.01 | 49.15 | 47.85 | 50.23 |
| 80 | 68.87 | 25.55 | 16.54 | 18.69 | 16.67 | 19.26 |
| 16 | 69.37 | 15.92 | -2.16 | -3.70 | -2.43 | 0.17 |
| 0 | 69.66 | 23.23 | -2.69 | -2.14 | -0.06 | 0.54 |

monochlorinated myricetin (formula II)+doxorubicin-STEDV

| Dox (nM) LB-1 (µM) | 100 | 20 | 4 | 0.8 | 0.16 | 0 |
|---|---|---|---|---|---|---|
| 10000 | 1.35 | 1.78 | 3.92 | 1.68 | 1.54 | 2.33 |
| 2000 | 1.03 | 1.95 | 1.48 | 0.16 | 0.98 | 2.53 |
| 400 | 1.15 | 2.18 | 4.18 | 2.69 | 2.55 | 2.25 |
| 80 | 0.81 | 1.12 | 4.04 | 1.76 | 2.76 | 2.75 |
| 16 | 2.45 | 4.18 | 2.48 | 2.37 | 1.07 | 2.78 |
| 0 | 1.14 | 3.02 | 2.79 | 1.15 | 4.29 | 1.43 |

The highest inhibition rate is 86.95%, at concentration of 100 µM LB-1 and 2000 nM doxorubicin.
The inhibition rate is 69.66% at 100 µM LB-1 only, and the inhibition rate is 51.6% at 2000 nM doxorubicin only.

TABLE 16

monochlorinated myricetin (formula II)+topotecan
monochlorinated myricetin (formula II)+topotecan-Relatively Cell inhibition of vehicle control(%)

| Top (nM) LB-1 (µM) | 100 | 20 | 4 | 0.8 | 0.16 | 0 |
|---|---|---|---|---|---|---|
| 10000 | 81.89 | 60.55 | 57.59 | 58.94 | 57.29 | 57.70 |
| 2000 | 97.18 | 60.03 | 58.19 | 59.40 | 57.25 | 58.27 |
| 400 | 81.95 | 48.35 | 47.20 | 45.93 | 45.53 | 45.72 |
| 80 | 70.93 | 35.49 | 29.47 | 28.43 | 28.07 | 29.61 |
| 16 | 70.31 | 21.80 | 1.58 | 0.89 | -0.41 | 2.49 |
| 0 | 69.54 | 25.74 | 2.82 | 2.26 | 0.02 | 2.00 |
| monochlorinated myricetin (formula II) + topotecan-STEDV | | | | | | |
| Top (nM) LB-1 (µM) | 100 | 20 | 4 | 0.8 | 0.16 | 0 |
| 10000 | 0.90 | 1.74 | 0.61 | 0.79 | 0.76 | 0.31 |
| 2000 | 0.19 | 0.37 | 1.81 | 0.53 | 0.86 | 0.78 |
| 400 | 0.35 | 1.09 | 2.19 | 0.61 | 2.29 | 2.28 |
| 80 | 0.49 | 1.26 | 4.00 | 1.86 | 1.77 | 0.84 |
| 16 | 0.74 | 0.63 | 2.77 | 1.78 | 3.24 | 1.79 |
| 0 | 2.95 | 2.35 | 3.66 | 4.45 | 1.54 | 1.68 |

The highest inhibition rate is 97.18%, at concentration of 100 µM LB-1 and 2000 nM topotecan.
The inhibition rate is 69.54% at 100 µM LB-1 only, and the inhibition rate is 58.27% at 2000 nM topotecan only.

TABLE 17

monochlorinated myricetin (formula II)+cisplatin
monochlorinated myricetin (formula II)+cisplatin-Relatively Cell inhibition of vehicle control(%)

| Cis (µM) LB-1 (µM) | 100 | 20 | 4 | 0.8 | 0.16 | 0 |
|---|---|---|---|---|---|---|
| 30 | 82.48 | 65.57 | 25.15 | 21.89 | 22.02 | 22.74 |
| 6 | 76.69 | 60.57 | 0.88 | -0.24 | -1.18 | -0.49 |
| 1.2 | 74.41 | 62.03 | -2.60 | -3.06 | -3.33 | -1.67 |
| 0.24 | 74.01 | 61.77 | -0.35 | -1.19 | -2.49 | 0.12 |
| 0.048 | 73.94 | 61.17 | 1.38 | 0.59 | -1.17 | 1.63 |
| 0 | 72.33 | 16.54 | 7.92 | -1.15 | 5.02 | 3.37 |

monochlorinated myricetin (formula II)+cisplatin-STEDV

| Cis (µM) LB-1 (µM) | 100 | 20 | 4 | 0.8 | 0.16 | 0 |
|---|---|---|---|---|---|---|
| 30 | 1.11 | 1.19 | 1.67 | 1.66 | 2.20 | 1.58 |
| 6 | 0.64 | 0.94 | 1.50 | 2.10 | 0.22 | 2.66 |
| 1.2 | 0.30 | 0.47 | 2.33 | 2.37 | 2.95 | 3.49 |
| 0.24 | 1.07 | 0.86 | 3.42 | 4.76 | 1.16 | 5.82 |
| 0.048 | 0.55 | 1.46 | 1.10 | 1.14 | 3.65 | 2.36 |
| 0 | 1.26 | 20.00 | 1.36 | 6.35 | 1.83 | 5.24 |

The highest inhibition rate is 82.48%, at concentration of 100 µM LB-1 and 30 µM cisplatin.
The inhibition rate is 72.33% at 100 µM LB-1 only, and the inhibition rate is 22.74% at 30 µM cisplatin only.

TABLE 18

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 44.40 | 46.51 | 44.58 | 47.33 | 47.66 | 45.79 |
| 200 | 41.08 | 46.14 | 45.88 | 47.93 | 49.27 | 48.51 |
| 40 | 44.36 | 47.48 | 48.55 | 50.52 | 49.39 | 48.57 |
| 8 | 39.39 | 39.68 | 38.38 | 42.48 | 39.90 | 40.81 |
| 1.6 | 32.51 | 13.65 | 12.76 | 17.65 | 18.07 | 16.56 |
| 0 | 30.03 | 5.67 | 0.20 | 6.31 | 4.71 | 5.19 |
| myricetin+taxol-STEDV | | | | | | |
| Tax (nM) Myr (µM) | 100 | 20 | 4 | 0.8 | 0.16 | 0 |
| 1000 | 3.61 | 0.27 | 2.04 | 2.00 | 2.24 | 0.59 |
| 200 | 1.47 | 0.90 | 1.10 | 1.30 | 1.00 | 1.61 |
| 40 | 0.95 | 0.22 | 1.58 | 0.60 | 2.19 | 2.27 |
| 8 | 4.50 | 3.06 | 3.95 | 0.81 | 2.43 | 1.31 |
| 1.6 | 1.97 | 3.38 | 2.30 | 2.08 | 2.24 | 4.72 |
| 0 | 1.66 | 2.62 | 7.98 | 1.59 | 4.37 | 2.64 |

The highest inhibition rate is 50.52%, at concentration of 0.8 µM myricetin and 40 nM taxol.
The inhibition rate is 6.31% at 0.8 µM myricetin only, and the inhibition rate is 48.57% at 40 nM taxol only.

TABLE 19

myricetin+doxorubicin
myricetin+doxorubicin-Relatively Cell inhibition of vehicle control(%)

| | | | | | | |
|---|---|---|---|---|---|---|
| 10000 | 61.75 | 51.39 | 49.82 | 51.79 | 48.81 | 48.11 |
| 2000 | 77.64 | 74.72 | 58.95 | 59.58 | 57.28 | 59.00 |
| 400 | 42.35 | 43.16 | 52.15 | 54.44 | 54.05 | 53.68 |
| 80 | 29.00 | 11.72 | 18.28 | 24.07 | 22.89 | 23.02 |
| 16 | 24.81 | 10.48 | 3.40 | 8.89 | 4.11 | 6.02 |
| 0 | 24.48 | 8.28 | 1.01 | 8.68 | 3.53 | 7.83 |

myricetin+doxorubicin-STEDV

| | | | | | | |
|---|---|---|---|---|---|---|
| Dox (nM) Myr (µM) | 100 | 20 | 4 | 0.8 | 0.16 | 0 |
| 10000 | 1.78 | 0.57 | 2.18 | 0.67 | 4.15 | 1.34 |
| 2000 | 1.97 | 1.17 | 3.44 | 1.16 | 1.82 | 1.05 |
| 400 | 1.57 | 1.85 | 4.18 | 4.39 | 2.19 | 1.32 |
| 80 | 2.43 | 2.56 | 5.73 | 2.68 | 2.29 | 2.01 |
| 16 | 4.12 | 4.49 | 6.22 | 2.82 | 3.62 | 2.63 |
| 0 | 1.85 | 2.18 | 3.30 | 2.50 | 1.48 | 2.28 |

The highest inhibition rate is 77.64%, at concentration of 100 µM myricetin and 2000 nM doxorubicin.
The inhibition rate is 24.48% at 100 µM myricetin only, and the inhibition rate is 59% at 2000 nM doxorubicin only.

TABLE 20

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 67.56 | 62.04 | 59.05 | 60.15 | 60.67 | 61.86 |
| 2000 | 64.18 | 61.77 | 60.82 | 63.00 | 63.61 | 62.70 |
| 400 | 54.78 | 50.20 | 48.41 | 51.27 | 51.8 | 53.94 |
| 80 | 42.73 | 36.11 | 36.22 | 37.88 | 38.8 | 36.93 |
| 16 | 29.35 | 13.35 | 8.65 | 10.23 | 12.71 | 12.5 |
| 0 | 22.21 | 3.54 | -2.25 | 1.93 | 3.07 | 2.32 |
| myricetin+topotecan-STEDV | | | | | | |
| Top (nM) Myr (µM) | 100 | 20 | 4 | 0.8 | 0.16 | 0 |
| 10000 | 1.59 | 1.05 | 1.99 | 0.74 | 2.09 | 2.51 |
| 2000 | 1.99 | 1.01 | 0.80 | 0.46 | 1.65 | 2.46 |
| 400 | 1.42 | 1.44 | 3.46 | 1.13 | 1.10 | 2.10 |
| 80 | 4.27 | 2.98 | 1.01 | 2.09 | 1.86 | 2.76 |
| 16 | 3.85 | 2.57 | 3.41 | 1.51 | 3.16 | 4.80 |
| 0 | 6.26 | 1.84 | 1.22 | 1.48 | 2.21 | 0.96 |

The highest inhibition rate is 67.56%, at concentration of 100 µM myricetin and 10000 nM topotecan.
The inhibition rate is 22.21% at 100 µM myricetin only, and the inhibition rate is 61.86% at 10000 nM topotecan only.

TABLE 21

myricetin+cisplatin
myricetin+cisplatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 53.89 | 39.18 | 34.26 | 37.94 | 38.10 | 36.54 |
| 6 | 36.66 | 16.98 | 11.67 | 11.62 | 12.66 | 11.28 |
| 1.2 | 30.07 | 0.18 | -3.61 | -2.81 | -1.33 | 0.07 |
| 0.24 | 28.57 | 1.89 | -6.14 | -2.46 | -1.29 | 0.20 |
| 0.048 | 21.65 | -2.79 | -8.40 | -0.55 | -1.23 | 0.23 |
| 0 | 25.35 | 2.08 | -0.88 | -1.72 | 2.98 | 2.91 |
| myricetin+cisplatin-STEDV | | | | | | |
| Cis (µM) Myr (µM) | 100 | 20 | 4 | 0.8 | 0.16 | 0 |
| 30 | 4.00 | 2.70 | 1.99 | 2.57 | 3.08 | 1.76 |
| 6 | 1.72 | 5.31 | 3.22 | 3.87 | 5.32 | 4.64 |
| 1.2 | 4.91 | 8.34 | 2.02 | 2.16 | 1.52 | 3.19 |
| 0.24 | 1.00 | 3.07 | 2.24 | 1.78 | 1.15 | 3.04 |
| 0.048 | 3.72 | 2.75 | 2.09 | 3.65 | 4.23 | 2.20 |
| 0 | 1.45 | 6.10 | 2.16 | 2.03 | 6.37 | 2.58 |

The highest inhibition rate is 53.89%, at concentration of 100 µM myricetin and 30 µM cisplatin.
The inhibition rate is 25.35% at 100 µM myricetin only, and the inhibition rate is 36.54% at 30 µM cisplatin only.

U87MG

TABLE 22

monochlorinated myricetin (formula II)+taxol
monochlorinated myricetin (formula II)+taxol-Relatively Cell inhibition of vehicle control(%)

| 1000 | 83.65 | 64.09 | 65.76 | 65.22 | 65.8 | 66.20 |
|---|---|---|---|---|---|---|
| 200 | 82.06 | 65.34 | 66.80 | 67.32 | 67.00 | 67.80 |
| 40 | 81.49 | 55.34 | 53.58 | 55.77 | 55.21 | 53.86 |
| 8 | 71.31 | 43.96 | 41.96 | 38.29 | 44.04 | 42.66 |
| 1.6 | 67.20 | 7.45 | 5.43 | 6.55 | 7.98 | 5.92 |
| 0 | 50.87 | 3.94 | 1.18 | 1.49 | 1.46 | -1.15 |
| monochlorinated myricetin (formula II)+taxol-STEDV | | | | | | |
| Tax (nM) LB-1 (µM) | 50 | 20 | 4 | 0.8 | 0.16 | 0 |
| 1000 | 0.69 | 1.17 | 0.52 | 0.64 | 0.77 | 2.55 |
| 200 | 1.22 | 1.91 | 1.60 | 1.10 | 0.30 | 2.26 |
| 40 | 1.10 | 1.04 | 3.23 | 1.25 | 0.49 | 0.87 |
| 8 | 2.64 | 2.98 | 2.18 | 2.90 | 0.71 | 2.89 |
| 1.6 | 1.67 | 3.92 | 3.70 | 1.95 | 2.71 | 5.22 |
| 0 | 2.58 | 3.07 | 2.15 | 4.84 | 1.49 | 3.73 |

The highest inhibition rate is 83.65%, at concentration of 50 µM LB-1 and 1000 nM taxol.
The inhibition rate is 50.87% at 50 µM LB-1 only, and the inhibition rate is 66.2% at 1000 nM taxol only.

TABLE 23

monochlorinated myricetin (formula II)+doxorubicin
monochlorinated myricetin (formula II)+doxorubicin-Relatively Cell inhibition of vehicle control(%)

| Dox (nM) LB-1 (µM) | 50 | 20 | 4 | 0.8 | 0.16 | 0 |
|---|---|---|---|---|---|---|
| 10000 | 92.37 | 77.88 | 77.93 | 77.88 | 78.27 | 78.41 |
| 2000 | 92.61 | 75.52 | 72.82 | 71.43 | 72.37 | 72.38 |
| 400 | 99.03 | 91.01 | 91.50 | 91.09 | 91.69 | 91.34 |
| 80 | 78.04 | 50.41 | 48.8 | 49.86 | 49.33 | 49.99 |
| 16 | 68.77 | 23.87 | 22.00 | 24.64 | 26.78 | 27.52 |
| 0 | 52.98 | 2.71 | 1.19 | 0.45 | 1.86 | 5.41 |
| monochlorinated myricetin (formula II)+doxorubicin-STEDV | | | | | | |
| Dox (nM) LB-1 (µM) | 50 | 20 | 4 | 0.8 | 0.16 | 0 |
| 10000 | 0.93 | 2.21 | 0.71 | 1.09 | 0.94 | 1.89 |
| 2000 | 0.37 | 0.62 | 0.82 | 2.24 | 0.32 | 1.00 |
| 400 | 0.09 | 0.13 | 0.46 | 0.36 | 0.41 | 0.15 |
| 80 | 1.56 | 2.15 | 2.91 | 3.22 | 1.39 | 2.06 |
| 16 | 0.78 | 0.93 | 2.28 | 2.67 | 1.84 | 2.07 |
| 0 | 2.09 | 4.11 | 2.80 | 8.89 | 4.35 | 0.83 |

The highest inhibition rate is 99.03%, at concentration of 50 µM LB-1 and 400 nM doxorubicin.
The inhibition rate is 52.98% at 50 µM LB-1 only, and the inhibition rate is 91.34% at 400 nM doxorubicin only.

TABLE 24

monochlorinated myricetin formula II)+topotecan
monochlorinated myricetin (formula II)+topotecan-Relatively Cell inhibition of vehicle control(%)

| Top (nM) LB-1 (µM) | 50 | 20 | 4 | 0.8 | 0.16 | 0 |
|---|---|---|---|---|---|---|
| 10000 | 96.03 | 83.73 | 82.29 | 82.53 | 82.91 | 82.10 |
| 2000 | 98.84 | 77.56 | 74.81 | 74.27 | 72.93 | 73.53 |
| 400 | 93.99 | 68.40 | 64.61 | 64.41 | 63.79 | 64.14 |
| 80 | 82.07 | 55.77 | 52.73 | 51.68 | 49.95 | 49.29 |
| 16 | 72.78 | 15.74 | 13.57 | 13.20 | 12.92 | 15.55 |
| 0 | 54.74 | 10.39 | 7.46 | 8.26 | 7.61 | 7.66 |
| monochlorinated myricetin (formula II)+topotecan-STEDV | | | | | | |
| Top (nM) LB-1 (µM) | 50 | 20 | 4 | 0.8 | 0.16 | 0 |
| 10000 | 0.20 | 0.93 | 0.94 | 0.75 | 1.08 | 0.78 |
| 2000 | 0.20 | 0.24 | 1.53 | 0.79 | 0.95 | 1.49 |
| 400 | 0.34 | 1.33 | 1.65 | 1.91 | 1.59 | 1.51 |
| 80 | 1.13 | 0.61 | 1.22 | 1.73 | 1.82 | 1.53 |
| 16 | 1.70 | 0.22 | 3.57 | 3.02 | 5.59 | 2.12 |
| 0 | 4.64 | 4.43 | 5.16 | 2.69 | 5.57 | 3.72 |

The highest inhibition rate is 98.84%, at concentration of 50 µM LB-1 and 2000 nM topotecan.
The inhibition rate is 54.74% at 50 µM LB-1 only, and the inhibition rate is 73.53% at 2000 nM topotecan only.

TABLE 25

monochlorinated myricetin (formula II)+cisplatin
monochlorinated myricetin (formula II)+cisplatin-Relatively Cell inhibition of vehicle control(%)

| Cis (µM) LB-1 (µM) | 50 | 20 | 4 | 0.8 | 0.16 | 0 |
|---|---|---|---|---|---|---|
| 30 | 95.97 | 84.60 | 82.00 | 79.78 | 82.53 | 83.20 |
| 6 | 88.51 | 49.50 | 45.98 | 43.82 | 46.61 | 46.78 |
| 1.2 | 79.49 | 14.76 | 10.40 | 11.98 | 14.69 | 11.78 |
| 0.24 | 68.91 | 4.13 | 5.65 | 5.35 | 6.2 | 3.89 |
| 0.048 | 67.39 | 2.54 | 2.21 | 4.62 | 6.68 | 1.97 |
| 0 | 52.31 | 1.46 | 0.32 | 1.81 | 3.45 | 1.99 |

monochlorinated myricetin (formula II)+cisplatin-STEDV

| Cis (µM) LB-1 (µM) | 50 | 20 | 4 | 0.8 | 0.16 | 0 |
|---|---|---|---|---|---|---|
| 30 | 0.20 | 0.18 | 1.41 | 4.10 | 0.74 | 1.54 |
| 6 | 0.69 | 0.46 | 2.57 | 4.74 | 1.06 | 1.11 |
| 1.2 | 0.34 | 1.60 | 2.28 | 1.73 | 3.06 | 1.18 |
| 0.24 | 2.40 | 6.11 | 1.37 | 0.34 | 2.87 | 0.99 |
| 0.048 | 0.69 | 3.18 | 4.84 | 4.26 | 2.45 | 1.64 |
| 0 | 1.79 | 5.06 | 3.06 | 1.82 | 3.15 | 2.92 |

The highest inhibition rate is 95.97%, at concentration of 50 µM LB-1 and 30 µM cisplatin.
The inhibition rate is 52.31% at 50 µM LB-1 only, and the inhibition rate is 83.2% at 30 µM cisplatin only.

TABLE 26

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 75.57 | 70.68 | 68.27 | 68.22 | 69.19 | 69.83 |
| 200 | 77.49 | 72.38 | 70.69 | 72.21 | 71.43 | 72.15 |
| 40 | 68.27 | 59.81 | 59.75 | 60.68 | 60.80 | 60.61 |
| 8 | 62.93 | 49.45 | 47.75 | 47.99 | 47.57 | 46.46 |
| 1.6 | 46.66 | 13.59 | 3.00 | 6.10 | 6.39 | 6.87 |
| 0 | 45.71 | 11.08 | 0.66 | 2.29 | 1.66 | 0.86 |
| myricetin+taxol-STEDV | | | | | | |
| Tax (nM) Myr (µM) | 100 | 20 | 4 | 0.8 | 0.16 | 0 |
| 1000 | 0.66 | 0.27 | 0.85 | 1.36 | 0.72 | 1.36 |
| 200 | 0.50 | 1.64 | 1.92 | 2.83 | 1.60 | 1.26 |
| 40 | 0.12 | 1.26 | 0.53 | 1.22 | 0.61 | 1.48 |
| 8 | 1.68 | 1.34 | 2.35 | 0.35 | 1.65 | 0.60 |
| 1.6 | 1.34 | 0.99 | 1.95 | 4.35 | 2.27 | 4.84 |
| 0 | 2.78 | 0.90 | 1.62 | 4.94 | 3.66 | 5.06 |

The highest inhibition rate is 77.49%, at concentration of 100 µM myricetin and 200 nM taxol.
The inhibition rate is 45.71% at 100 µM myricetin only, and the inhibition rate is 72.15% at 200 nM taxol only.

TABLE 27

myricetin+doxorubicin
myricetin+doxorubicin-Relatively Cell inhibition of vehicle control(%)

| | | | | | | |
|---|---|---|---|---|---|---|
| Dox (nM) Myr (µM) | 100 | 20 | 4 | 0.8 | 0.16 | 0 |
| 10000 | 83.61 | 82.21 | 81.82 | 81.41 | 80.83 | 80.81 |
| 2000 | 92.56 | 79.01 | 77.20 | 77.61 | 77.89 | 78.32 |
| 400 | 73.66 | 79.34 | 90.16 | 92.28 | 93.15 | 93.04 |
| 80 | 60.46 | 47.44 | 50.52 | 53.02 | 52.96 | 52.53 |
| 16 | 50.56 | 24.57 | 23.38 | 25.27 | 26.25 | 27.36 |
| 0 | 32.30 | 7.98 | 1.02 | -1.19 | 0.21 | 3.15 |

myricetin+doxorubicin-STEDV

| Dox (nM) Myr (µM) | 100 | 20 | 4 | 0.8 | 0.16 | 0 |
|---|---|---|---|---|---|---|
| 10000 | 0.65 | 0.59 | 0.59 | 0.97 | 0.11 | 1.56 |
| 2000 | 1.63 | 1.04 | 3.90 | 1.95 | 0.97 | 0.63 |
| 400 | 0.73 | 0.85 | 0.40 | 0.72 | 0.21 | 0.19 |
| 80 | 1.08 | 1.76 | 0.86 | 0.46 | 0.71 | 1.28 |
| 16 | 2.05 | 1.19 | 2.69 | 1.98 | 1.79 | 2.48 |
| 0 | 26.64 | 2.10 | 1.67 | 1.46 | 2.76 | 3.15 |

The highest inhibition rate is 93.15%, at concentration of 0.16 µM myricetin and 400 nM doxorubicin.
The inhibition rate is 0.21% at 0.16 µM myricetin only, and the inhibition rate is 93.04% at 400 nM doxorubicin only.

TABLE 28

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 87.63 | 84.07 | 84.24 | 83.84 | 83.75 | 83.44 |
| 2000 | 82.40 | 76.64 | 75.82 | 76.93 | 76.62 | 76.31 |
| 400 | 74.84 | 67.67 | 66.83 | 66.37 | 66.31 | 66.22 |
| 80 | 67.02 | 54.39 | 49.85 | 49.68 | 50.76 | 51.61 |
| 16 | 47.44 | 13.93 | 8.99 | 9.82 | 11.54 | 11.44 |
| 0 | 43.43 | 8.45 | 3.52 | 3.94 | 6.07 | 3.63 |
| myricetin+topotecan-STEDV | | | | | | |
| Top (nM) Myr (µM) | 100 | 20 | 4 | 0.8 | 0.16 | 0 |
| 10000 | 0.84 | 1.18 | 0.46 | 0.24 | 0.65 | 0.19 |
| 2000 | 0.93 | 0.56 | 0.75 | 0.63 | 0.94 | 0.87 |
| 400 | 0.79 | 0.51 | 0.84 | 1.24 | 0.69 | 0.61 |
| 80 | 2.40 | 0.89 | 1.37 | 2.77 | 1.46 | 0.69 |
| 16 | 2.89 | 4.63 | 1.53 | 5.96 | 2.32 | 3.72 |
| 0 | 4.60 | 1.44 | 3.57 | 1.84 | 1.20 | 4.07 |

The highest inhibition rate is 87.63%, at concentration of 100 µM myricetin and 10000 nM topotecan.
The inhibition rate is 43.43% at 100 µM myricetin only, and the inhibition rate is 83.44% at 10000 nM topotecan only.

TABLE 29

myricetin+cisplatin
myricetin+cisplatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 90.10 | 86.91 | 85.71 | 84.79 | 85.14 | 86.28 |
| 6 | 68.78 | 59.84 | 57.94 | 57.50 | 57.55 | 58.90 |
| 1.2 | 50.05 | 14.92 | 11.74 | 13.71 | 12.63 | 15.05 |
| 0.24 | 43.74 | 4.86 | 1.37 | 2.02 | 0.83 | 1.32 |
| 0.048 | 43.41 | 5.35 | -0.21 | -0.06 | -2.78 | 0.89 |
| 0 | 46.84 | 9.21 | -0.71 | 1.25 | 0.23 | -0.52 |
| myricetin+cisplatin-STEDV | | | | | | |
| Cis (µM) Myr (µM) | 100 | 20 | 4 | 0.8 | 0.16 | 0 |
| 30 | 0.27 | 0.33 | 0.12 | 1.68 | 0.49 | 0.90 |
| 6 | 0.57 | 2.47 | 2.66 | 1.66 | 0.51 | 2.45 |
| 1.2 | 1.13 | 1.84 | 4.84 | 0.55 | 1.89 | 2.39 |
| 0.24 | 2.88 | 4.26 | 1.44 | 3.44 | 1.42 | 0.84 |
| 0.048 | 1.43 | 2.57 | 1.84 | 1.68 | 3.42 | 2.62 |
| 0 | 3.41 | 4.17 | 7.85 | 2.98 | 6.57 | 1.19 |

The highest inhibition rate is 90.10%, at concentration of 100 µM myricetin and 30 µM cisplatin.
The inhibition rate is 46.84% at 100 µM myricetin only, and the inhibition rate is 86.28% at 30 µM cisplatin only.

LNCaP

TABLE 30

monochlorinated myricetin formula II+taxol
monochlorinated myricetin (formula II)+taxol-Relatively Cell inhibition of vehicle control(%)

| Tax (nM) LB-1 (µM) | 50 | 20 | 4 | 0.8 | 0.16 | 0 |
|---|---|---|---|---|---|---|
| 1000 | 98.57 | 95.65 | 61.87 | 49.91 | 50.29 | 48.77 |
| 200 | 98.29 | 95.39 | 60.85 | 56.98 | 56.60 | 56.42 |
| 40 | 98.25 | 95.00 | 60.80 | 58.19 | 59.92 | 59.18 |
| 8 | 98.26 | 94.69 | 57.99 | 56.38 | 54.96 | 53.97 |
| 1.6 | 97.78 | 93.92 | 38.99 | 15.47 | 17.11 | 9.88 |
| 0 | 97.55 | 93.38 | 39.06 | 9.07 | 13.36 | 3.01 |
| monochlorinated myricetin (formula II)+taxol-STEDV | | | | | | |
| Tax (nM) LB-1 (µM) | 50 | 20 | 4 | 0.8 | 0.16 | 0 |
| 1000 | 0.21 | 0.55 | 2.21 | 0.75 | 1.03 | 0.76 |
| 200 | 0.06 | 0.39 | 1.29 | 1.91 | 2.97 | 2.65 |
| 40 | 0.26 | 0.53 | 0.27 | 0.20 | 0.36 | 1.14 |
| 8 | 0.15 | 0.23 | 0.55 | 1.85 | 1.63 | 3.33 |
| 1.6 | 0.41 | 0.16 | 4.21 | 4.54 | 1.78 | 1.43 |
| 0 | 0.27 | 0.57 | 1.57 | 5.10 | 6.24 | 5.26 |

The highest inhibition rate is 98.57%, at concentration of 50 µM LB-1 and 1000 nM taxol.
The inhibition rate is 97.55% at 50 µM LB-1 only, and the inhibition rate is 48.77% at 1000 nM taxol only.

TABLE 31

monochlorinated myricetin (formula II)+doxorubicin
monochlorinated myricetin (formula II)+doxorubicin-Relatively Cell inhibition of vehicle control(%)

| Dox (nM) LB-1 (µM) | 50 | 20 | 4 | 0.8 | 0.16 | 0 |
|---|---|---|---|---|---|---|
| 10000 | 94.43 | 92.72 | 88.76 | 88.35 | 87.88 | 87.77 |
| 2000 | 99.42 | 98.41 | 98.20 | 97.71 | 97.73 | 97.20 |
| 400 | 99.78 | 99.26 | 74.28 | 67.97 | 67.68 | 65.15 |
| 80 | 98.61 | 96.43 | 48.72 | 32.30 | 33.44 | 29.95 |
| 16 | 97.46 | 93.88 | 36.59 | 7.73 | 11.31 | 7.17 |
| 0 | 97.55 | 93.38 | 43.01 | 10.83 | 11.64 | 3.91 |
| monochlorinated myricetin (formula II)+doxorubicin-STEDV | | | | | | |
| Dox (nM) LB-1 (µM) | 50 | 20 | 4 | 0.8 | 0.16 | 0 |
| 10000 | 0.54 | 0.25 | 0.40 | 1.24 | 0.83 | 0.65 |
| 2000 | 0.12 | 0.14 | 0.04 | 0.30 | 0.32 | 0.10 |
| 400 | 0.03 | 0.12 | 2.09 | 1.53 | 0.88 | 2.07 |
| 80 | 0.18 | 0.24 | 2.10 | 4.35 | 1.07 | 2.36 |
| 16 | 0.31 | 0.94 | 0.47 | 5.83 | 1.22 | 3.74 |
| 0 | 0.31 | 0.86 | 2.55 | 5.76 | 4.29 | 3.94 |

The highest inhibition rate is 99.78%, at concentration of 50 µM LB-1 and 400 nM doxorubicin.
The inhibition rate is 97.55% at 50 µM LB-1 only, and the inhibition rate is 65.15% at 400 nM doxorubicin only.

TABLE 32

monochlorinated myricetin (formula II)+topotecan
monochlorinated myricetin (formula II)+topotecan-Relatively Cell inhibition of vehicle control(%)

| Top (nM) LB-1 (µM) | 50 | 20 | 4 | 0.8 | 0.16 | 0 |
|---|---|---|---|---|---|---|
| 10000 | 92.66 | 93.06 | 88.69 | 84.59 | 84.34 | 84.79 |
| 2000 | 99.82 | 99.62 | 93.85 | 84.44 | 83.26 | 82.06 |
| 400 | 99.61 | 99.01 | 67.76 | 60.84 | 58.54 | 55.90 |
| 80 | 98.33 | 95.72 | 49.94 | 40.78 | 41.10 | 37.76 |
| 16 | 97.57 | 94.55 | 41.81 | 17.79 | 21.15 | 15.36 |
| 0 | 97.57 | 93.59 | 38.05 | 16.26 | 10.46 | 1.39 |
| monochlorinated myricetin (formula II)+topotecan-STEDV | | | | | | |
| Top (nM) LB-1(µM) | 50 | 20 | 4 | 0.8 | 0.16 | 0 |
| 10000 | 0.15 | 0.45 | 0.36 | 0.31 | 0.61 | 0.11 |
| 2000 | 0.01 | 0.02 | 0.19 | 1.40 | 0.99 | 0.48 |
| 400 | 0.09 | 0.05 | 1.34 | 1.03 | 1.59 | 0.93 |
| 80 | 0.32 | 0.35 | 2.94 | 4.19 | 1.38 | 1.13 |
| 16 | 0.25 | 0.31 | 2.91 | 2.25 | 1.42 | 1.37 |
| 0 | 0.21 | 0.33 | 2.91 | 0.60 | 5.91 | 8.28 |

The highest inhibition rate is 99.82%, at concentration of 50 µM LB-1 and 2000 nM topotecan.
The inhibition rate is 97.57% at 50 µM LB-1 only, and the inhibition rate is 82.06% at 2000 nM topotecan only.

TABLE 33

monochlorinated myricetin (formula II)+cisplatin
monochlorinated myricetin (formula II)+cisplatin-Relatively Cell inhibition of vehicle control(%)

| Cis (µM) LB-1 (µM) | 50 | 20 | 4 | 0.8 | 0.16 | 0 |
|---|---|---|---|---|---|---|
| 30 | 99.48 | 99.00 | 73.22 | 46.34 | 46.03 | 53.09 |
| 6 | 99.14 | 96.19 | 46.99 | 14.93 | 13.32 | 12.99 |
| 1.2 | 98.06 | 93.53 | 31.47 | -10.1 | -7.20 | -13.28 |
| 0.24 | 97.37 | 93.31 | 31.21 | -11.89 | -11.6 | -20.44 |
| 0.048 | 97.28 | 93.66 | 35.81 | -4.16 | -2.85 | -11.07 |
| 0 | 97.41 | 93.13 | 40.00 | 7.03 | 11.16 | 3.79 |
| monochlorinated myricetin (formula II)+cisplatin-STEDV | | | | | | |
| Cis (µM) LB-1 (µM) | 50 | 20 | 4 | 0.8 | 0.16 | 0 |
| 30 | 0.01 | 0.17 | 4.36 | 4.63 | 3.59 | 4.98 |
| 6 | 0.08 | 0.37 | 4.74 | 2.00 | 2.40 | 2.77 |
| 1.2 | 0.16 | 0.35 | 6.52 | 3.50 | 2.83 | 2.30 |
| 0.24 | 0.29 | 0.32 | 7.79 | 3.83 | 6.13 | 10.04 |
| 0.048 | 0.11 | 0.35 | 3.13 | 2.84 | 6.36 | 5.72 |
| 0 | 0.27 | 0.27 | 2.97 | 5.70 | 6.74 | 9.60 |

The highest inhibition rate is 99.48%, at concentration of 50 µM LB-1 and 30 µM cisplatin.
The inhibition rate is 97.41% at 50 µM LB-1 only, and the inhibition rate is 53.09% at 30 µM cisplatin only.

TABLE 34

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 58.26 | 55.35 | 54.76 | 57.41 | 56.90 | 55.57 |
| 200 | 61.69 | 59.23 | 61.56 | 63.61 | 63.04 | 62.20 |
| 40 | 62.15 | 61.65 | 63.90 | 65.76 | 64.28 | 65.51 |
| 8 | 61.90 | 59.98 | 61.17 | 62.97 | 62.75 | 62.64 |
| 1.6 | 44.22 | 18.73 | 11.87 | 11.54 | 13.03 | 12.14 |
| 0 | 31.42 | 4.80 | -5.50 | -2.06 | -2.95 | 1.23 |
| myricetin+taxol-STEDV | | | | | | |
| Tax (nM) Myr (µM) | 100 | 20 | 4 | 0.8 | 0.16 | 0 |
| 1000 | 0.80 | 0.49 | 1.68 | 2.15 | 1.55 | 0.62 |
| 200 | 0.38 | 0.84 | 1.80 | 0.20 | 1.50 | 1.29 |
| 40 | 0.78 | 1.17 | 1.50 | 1.44 | 1.12 | 0.51 |
| 8 | 3.62 | 1.09 | 0.89 | 0.97 | 1.18 | 1.29 |
| 1.6 | 4.05 | 3.28 | 3.72 | 2.77 | 1.94 | 1.12 |
| 0 | 6.17 | 5.00 | 6.00 | 3.94 | 4.60 | 4.69 |

The highest inhibition rate is 65.76%, at concentration of 0.8 µM myricetin and 40 nM taxol.
The inhibition rate is -2.06% at 0.8 µM myricetin only, and the inhibition rate is 65.51% at 40 nM taxol only.

TABLE 35

myricetin+doxorubicin
myricetin+doxorubicin-Relatively Cell inhibition of vehicle control(%)

| Dox (nM) Myr (µM) | 100 | 20 | 4 | 0.8 | 0.16 | 0 |
|---|---|---|---|---|---|---|
| 10000 | 89.89 | 88.99 | 89.19 | 88.99 | 89.03 | 88.88 |
| 2000 | 94.38 | 97.50 | 97.73 | 97.01 | 97.05 | 96.97 |
| 400 | 59.42 | 56.07 | 64.40 | 68.79 | 69.46 | 67.33 |
| 80 | 36.87 | 30.71 | 31.63 | 31.39 | 34.65 | 31.68 |
| 16 | 27.32 | 11.29 | 5.71 | 0.04 | -0.74 | 2.69 |
| 0 | 21.11 | -2.66 | -3.26 | -0.32 | -12.67 | -2.96 |
| myricetin+doxorubicin-STEDV | | | | | | |
| Dox (nM) Myr (µM) | 100 | 20 | 4 | 0.8 | 0.16 | 0 |
| 10000 | 0.14 | 0.34 | 0.41 | 1.28 | 0.83 | 1.09 |
| 2000 | 0.48 | 0.33 | 0.43 | 0.66 | 0.40 | 0.58 |
| 400 | 1.98 | 2.00 | 1.99 | 0.92 | 0.85 | 0.47 |
| 80 | 5.71 | 2.66 | 4.58 | 4.11 | 5.16 | 4.60 |
| 16 | 9.49 | 4.84 | 4.64 | 6.00 | 5.85 | 4.16 |
| 0 | 4.83 | 5.13 | 4.28 | 0.86 | 11.00 | 9.28 |

The highest inhibition rate is 97.73%, at concentration of 4 µM myricetin and 2000 nM doxorubicin.
The inhibition rate is -3.26% at 4 µM myricetin only, and the inhibition rate is 96.97% at 2000 nM doxorubicin only.

TABLE 36

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 91.49 | 88.76 | 88.3 | 87.86 | 87.62 | 87.46 |
| 2000 | 90.37 | 86.82 | 84.4 | 84.15 | 84.14 | 83.62 |
| 400 | 72.56 | 61.66 | 61.77 | 61.86 | 62.67 | 59.99 |
| 80 | 62.16 | 44.39 | 44.18 | 41.02 | 43.16 | 40.13 |
| 16 | 48.08 | 28.15 | 21.41 | 13.85 | 17.08 | 17.61 |
| 0 | 30.08 | 6.50 | 8.50 | -2.79 | -2.14 | -1.30 |
| myricetin+topotecan-STEDV | | | | | | |
| Top (nM) Myr (µM) | 100 | 20 | 4 | 0.8 | 0.16 | 0 |
| 10000 | 0.25 | 0.08 | 0.52 | 0.89 | 0.03 | 0.62 |
| 2000 | 0.59 | 0.11 | 0.69 | 1.29 | 0.95 | 1.19 |
| 400 | 0.83 | 1.29 | 1.76 | 0.81 | 1.68 | 1.35 |
| 80 | 0.49 | 1.79 | 2.47 | 1.65 | 1.51 | 1.49 |
| 16 | 1.23 | 1.40 | 1.16 | 1.85 | 5.04 | 1.01 |
| 0 | 3.53 | 1.93 | 1.70 | 3.48 | 6.87 | 2.00 |

The highest inhibition rate is 91.49%, at concentration of 100 µM myricetin and 10000 nM topotecan.
The inhibition rate is 30.08% at 100 µM myricetin only, and the inhibition rate is 87.46% at 10000 nM topotecan only.

TABLE 37

myricetin+cisplatin
myricetin+cisplatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 77.31 | 67.89 | 59.21 | 62.25 | 63.08 | 61.54 |
| 6 | 49.22 | 26.74 | 20.08 | 18.87 | 19.76 | 19.01 |
| 1.2 | 32.80 | 0.25 | -7.76 | -16.51 | -7.78 | -9.21 |
| 0.24 | 25.64 | -2.42 | -12.06 | -19.28 | -22.31 | -17.2 |
| 0.048 | 28.68 | -0.03 | -6.72 | -18.4 | -16.73 | -7.42 |
| 0 | 27.19 | -3.11 | 0.26 | -1.65 | -2.76 | -0.33 |
| myricetin+cisplatin-STEDV | | | | | | |
| Cis (µM) Myr (µM) | 100 | 20 | 4 | 0.8 | 0.16 | 0 |
| 30 | 0.84 | 3.97 | 5.87 | 2.94 | 2.02 | 0.68 |
| 6 | 3.55 | 1.58 | 2.22 | 1.13 | 3.20 | 1.88 |
| 1.2 | 2.81 | 3.41 | 2.27 | 3.44 | 4.28 | 1.04 |
| 0.24 | 1.79 | 5.36 | 3.03 | 6.62 | 4.72 | 2.71 |
| 0.048 | 6.68 | 6.91 | 3.44 | 3.48 | 4.75 | 4.17 |
| 0 | 3.47 | 7.14 | 8.75 | 3.32 | 2.18 | 2.29 |

The highest inhibition rate is 77.31%, at concentration of 100 µM myricetin and 30 µM cisplatin.
The inhibition rate is 27.19% at 100 µM myricetin only, and the inhibition rate is 61.54% at 30 µM cisplatin only.

HT29

TABLE 38

monochlorinated myricetin (formula II)+taxol
monochlorinated myricetin (formula II)+taxol-Relatively Cell inhibition of vehicle control(%)

| Tax (nM) LB-1 (µM) | 100 | 20 | 4 | 0.8 | 0.16 | 0 |
|---|---|---|---|---|---|---|
| 1000 | 87.34 | 80.53 | 82.92 | 83.21 | 82.26 | 82.00 |
| 200 | 84.91 | 80.63 | 82.73 | 82.70 | 82.55 | 83.13 |
| 40 | 83.64 | 83.31 | 83.12 | 82.75 | 82.75 | 83.37 |
| 8 | 79.18 | 67.11 | 73.48 | 74.06 | 73.74 | 67.89 |
| 1.6 | 77.70 | 14.95 | 8.79 | 5.01 | 8.02 | 5.54 |
| 0 | 69.26 | 1.61 | -12.45 | -18.43 | -15.5 | -14.96 |
| monochlorinated myricetin (formula II)+taxol-STEDV | | | | | | |
| Tax (nM) LB-1 (µM) | 100 | 20 | 4 | 0.8 | 0.16 | 0 |
| 1000 | 1.09 | 1.52 | 1.67 | 1.92 | 1.81 | 1.44 |
| 200 | 3.23 | 1.97 | 2.55 | 2.91 | 2.74 | 1.80 |
| 40 | 4.25 | 2.04 | 1.16 | 1.52 | 1.63 | 0.87 |
| 8 | 7.05 | 2.15 | 2.30 | 3.49 | 2.80 | 2.89 |
| 1.6 | 0.16 | 1.10 | 4.87 | 5.22 | 0.75 | 4.42 |
| 0 | 2.51 | 3.39 | 7.63 | 9.41 | 7.07 | 7.40 |

The highest inhibition rate is 87.34%, at concentration of 50 µM LB-1 and 1000 nM taxol.
The inhibition rate is 69.26% at 50 µM LB-1 only, and the inhibition rate is 82% at 1000 nM taxol only.

TABLE 39

monochlorinated myricetin (formula II)+doxorubicin
monochlorinated myricetin (formula II)+doxorubicin-Relatively Cell inhibition of vehicle control(%)

| Dox (nM) LB-1 (µM) | 100 | 20 | 4 | 0.8 | 0.16 | 0 |
|---|---|---|---|---|---|---|
| 10000 | 87.60 | 82.14 | 80.34 | 79.05 | 79.44 | 79.09 |
| 2000 | 92.45 | 95.77 | 94.17 | 93.37 | 93.88 | 92.26 |
| 400 | 85.13 | 56.78 | 61.91 | 59.08 | 58.72 | 61.09 |
| 80 | 80.90 | 33.68 | 29.85 | 25.92 | 22.41 | 25.6 |
| 16 | 78.32 | 8.75 | 6.29 | 1.96 | 1.34 | 2.75 |
| 0 | 80.21 | 15.57 | 4.13 | 5.54 | 3.50 | 4.44 |
| monochlorinated myricetin (formula II)+doxorubicin-STEDV | | | | | | |
| Dox (nM) LB-1 (µM) | 100 | 20 | 4 | 0.8 | 0.16 | 0 |
| 10000 | 0.64 | 0.59 | 0.13 | 2.06 | 0.67 | 1.27 |
| 2000 | 0.53 | 0.07 | 0.40 | 1.09 | 0.79 | 1.18 |
| 400 | 1.96 | 0.87 | 1.90 | 1.37 | 2.82 | 0.33 |
| 80 | 3.19 | 3.43 | 4.99 | 3.92 | 5.88 | 2.34 |
| 16 | 2.00 | 3.30 | 6.39 | 3.51 | 2.42 | 4.66 |
| 0 | 1.54 | 5.06 | 0.98 | 3.09 | 1.40 | 4.64 |

The highest inhibition rate is 95.77%, at concentration of 20 µM LB-1 and 2000 nM doxorubicin.
The inhibition rate is 15.57% at 20 µM LB-1 only, and the inhibition rate is 92.26% at 2000 nM doxorubicin only.

TABLE 40

monochlorinated myricetin (formula II)+topotecan
monochlorinated myricetin (formula II)+topotecan-Relatively Cell inhibition of vehicle control(%)

| Top (nM) LB-1 (µM) | 100 | 20 | 4 | 0.8 | 0.16 | 0 |
|---|---|---|---|---|---|---|
| 10000 | 85.06 | 80.19 | 78.82 | 78.17 | 78.67 | 79.44 |
| 2000 | 98.29 | 95.29 | 93.28 | 93.65 | 93.5 | 93.16 |
| 400 | 88.78 | 68.16 | 67.78 | 66.64 | 67.02 | 64.79 |
| 80 | 79.94 | 31.31 | 26.90 | 25.50 | 24.56 | 26.11 |
| 16 | 78.34 | 18.76 | 10.22 | 13.14 | 12.19 | 12.50 |
| 0 | 69.73 | 13.78 | 2.19 | -2.82 | -1.89 | -3.42 |
| monochlorinated myricetin (formula II)+topotecan-STEDV | | | | | | |
| Top (nM) LB-1 (µM) | 100 | 20 | 4 | 0.8 | 0.16 | 0 |
| 10000 | 0.57 | 0.47 | 0.72 | 0.20 | 1.20 | 0.52 |
| 2000 | 0.24 | 0.42 | 1.04 | 0.56 | 0.59 | 0.30 |
| 400 | 0.89 | 0.50 | 0.49 | 1.91 | 0.72 | 1.78 |
| 80 | 1.13 | 4.89 | 5.06 | 4.87 | 3.30 | 3.81 |
| 16 | 0.55 | 4.74 | 4.56 | 4.26 | 7.05 | 7.10 |
| 0 | 19.26 | 11.24 | 0.84 | 1.93 | 2.01 | 2.77 |

The highest inhibition rate is 98.29%, at concentration of 50 µM LB-1 and 2000 nM topotecan.
The inhibition rate is 69.73% at 50 µM LB-1 only, and the inhibition rate is 93.16% at 2000 nM topotecan only.

TABLE 41

monochlorinated myricetin (formula II)+cisplatin
monochlorinated myricetin (formula II)+cisplatin-Relatively Cell inhibition of vehicle control(%)

| Cis (µM) LB-1 (µM) | 100 | 20 | 4 | 0.8 | 0.16 | 0 |
|---|---|---|---|---|---|---|
| 30 | 94.01 | 77.29 | 65.44 | 49.7 | 51.48 | 52.83 |
| 6 | 71.29 | 49.12 | 32.23 | 17.46 | 15.14 | 19.05 |
| 1.2 | 65.02 | 44.79 | 19.55 | 2.08 | 3.81 | 6.85 |
| 0.24 | 73.23 | 38.31 | 16.43 | -3.69 | -2.63 | 2.41 |
| 0.048 | 68.83 | 36.99 | 16.02 | -6.95 | -6.67 | 2.74 |
| 0 | 65.11 | 31.90 | 15.95 | -6.13 | -2.64 | 2.25 |
| monochlorinated myricetin (formula II)+cisplatin-STEDV | | | | | | |
| Cis (µM) LB-1 (µM) | 100 | 20 | 4 | 0.8 | 0.16 | 0 |
| 30 | 2.13 | 12.17 | 11.72 | 12.20 | 13.82 | 16.20 |
| 6 | 10.70 | 35.78 | 24.88 | 3.13 | 3.73 | 2.46 |
| 1.2 | 12.44 | 34.78 | 10.90 | 21.55 | 14.20 | 13.95 |
| 0.24 | 3.34 | 31.56 | 12.53 | 24.99 | 17.34 | 14.19 |
| 0.048 | 3.73 | 29.46 | 13.34 | 18.93 | 15.44 | 6.40 |
| 0 | 7.54 | 31.55 | 22.44 | 9.46 | 6.00 | 6.08 |

The highest inhibition rate is 94.01%, at concentration of 50 µM LB-1 and 30 µM cisplatin.
The inhibition rate is 65.11% at 50 µM LB-1 only, and the inhibition rate is 52.83% at 30 µM cisplatin only.

TABLE 42

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 76.26 | 74.35 | 81.07 | 80.15 | 80.50 | 79.03 |
| 200 | 84.30 | 74.74 | 82.66 | 81.61 | 81.29 | 81.35 |
| 40 | 96.99 | 70.90 | 81.23 | 81.90 | 81.78 | 80.46 |
| 8 | 97.60 | 65.68 | 69.59 | 72.77 | 74.44 | 73.73 |
| 1.6 | 85.95 | 31.95 | 11.98 | 9.26 | 14.54 | 3.93 |
| 0 | 88.39 | 32.84 | 0.80 | -1.36 | 1.65 | -3.26 |
| myricetin+taxol-STEDV | | | | | | |
| Tax (nM) r (µM) | 100 | 20 | 4 | 0.8 | 0.16 | 0 |
| 1000 | 9.91 | 1.82 | 0.84 | 0.53 | 0.68 | 1.30 |
| 200 | 3.05 | 1.76 | 1.62 | 2.48 | 2.27 | 1.73 |
| 40 | 0.61 | 0.87 | 1.14 | 0.62 | 0.30 | 0.85 |
| 8 | 0.25 | 3.10 | 2.82 | 3.29 | 1.32 | 6.72 |
| 1.6 | 4.74 | 8.61 | 7.43 | 3.44 | 6.12 | 3.27 |
| 0 | 7.17 | 11.70 | 6.10 | 1.61 | 4.37 | 1.91 |

The highest inhibition rate is 97.6%, at concentration of 100 µM myricetin and 8 nM taxol.
The inhibition rate is 88.39% at 100 µM myricetin only, and the inhibition rate is 73.73% at 8 nM taxol only.

TABLE 43

myricetin+doxorubicin
myricetin+doxorubicin-Relatively Cell inhibition of vehicle control(%)

| Dox (nM) Myr (µM) | 100 | 20 | 4 | 0.8 | 0.16 | 0 |
|---|---|---|---|---|---|---|
| 10000 | 91.16 | 87.40 | 84.81 | 83.46 | 83.92 | 82.88 |
| 2000 | 89.86 | 90.96 | 93.72 | 91.47 | 91.88 | 91.27 |
| 400 | 72.07 | 48.00 | 48.47 | 51.83 | 51.53 | 52.68 |
| 80 | 66.99 | 43.00 | 29.38 | 26.48 | 28.29 | 28.95 |
| 16 | 71.97 | 19.56 | 1.13 | -0.88 | 0.63 | -0.43 |
| 0 | 74.39 | 23.37 | -0.67 | -1.09 | -0.48 | -1.00 |
| myricetin+doxorubicin-STEDV | | | | | | |
| Dox (nM) Myr (µM) | 100 | 20 | 4 | 0.8 | 0.16 | 0 |
| 10000 | 0.42 | 0.83 | 0.78 | 0.20 | 0.79 | 1.80 |
| 2000 | 1.63 | 2.04 | 0.92 | 0.85 | 0.42 | 1.21 |
| 400 | 8.95 | 1.81 | 2.32 | 2.30 | 1.44 | 1.01 |
| 80 | 8.17 | 4.24 | 7.99 | 7.98 | 1.93 | 5.06 |
| 16 | 1.55 | 1.42 | 4.31 | 7.30 | 6.35 | 12.20 |
| 0 | 2.37 | 5.58 | 3.29 | 6.95 | 0.87 | 4.53 |

The highest inhibition rate is 93.72%, at concentration of 4 µM myricetin and 2000 nM doxorubicin.
The inhibition rate is -0.67% at 4 µM myricetin only, and the inhibition rate is 91.27% at 2000 nM doxorubicin only.

TABLE 44

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 94.00 | 85.95 | 80.93 | 81.06 | 81.28 | 82.42 |
| 2000 | 95.95 | 95.27 | 94.78 | 94.86 | 94.91 | 94.54 |
| 400 | 93.01 | 73.77 | 71.23 | 70.85 | 72.49 | 71.12 |
| 80 | 88.63 | 36.66 | 27.81 | 25.96 | 24.73 | 21.57 |
| 16 | 86.70 | 28.79 | 12.33 | 10.83 | 7.90 | 11.50 |
| 0 | 87.04 | 24.77 | 3.33 | -0.48 | -1.11 | -4.35 |
| myricetin+topotecan-STEDV | | | | | | |
| Top (nM) r (µM) | 100 | 20 | 4 | 0.8 | 0.16 | 0 |
| 10000 | 0.75 | 3.09 | 0.96 | 1.63 | 2.02 | 0.70 |
| 2000 | 0.29 | 0.43 | 1.10 | 0.76 | 0.43 | 0.46 |
| 400 | 0.37 | 1.02 | 2.17 | 1.30 | 3.29 | 1.74 |
| 80 | 2.47 | 7.23 | 4.31 | 6.62 | 4.31 | 4.05 |
| 16 | 0.99 | 4.94 | 5.25 | 5.65 | 6.08 | 5.14 |
| 0 | 0.83 | 1.26 | 4.02 | 3.23 | 7.90 | 2.14 |

The highest inhibition rate is 95.95%, at concentration of 100 µM myricetin and 2000 nM topotecan.
The inhibition rate is 87.04% at 100 µM myricetin only, and the inhibition rate is 94.54% at 2000 nM topotecan only.

TABLE 45

myricetin+cisplatin
myricetin+cisplatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 95.37 | 87.70 | 79.66 | 68.99 | 70.48 | 66.86 |
| 6 | 91.95 | 41.80 | 29.42 | 26.46 | 23.68 | 20.8 |
| 1.2 | 88.83 | 27.41 | 9.13 | 6.66 | 8.14 | 6.63 |
| 0.24 | 91.63 | 25.49 | 4.87 | 0.81 | 2.16 | 0.79 |
| 0.048 | 88.18 | 21.86 | 1.88 | 0.44 | -0.79 | -4.21 |
| 0 | 90.42 | 24.55 | 6.56 | 3.87 | 0.64 | 1.41 |

myricetin+cisplatin-STEDV

| Cis (µM) r (µM) | 100 | 20 | 4 | 0.8 | 0.16 | 0 |
|---|---|---|---|---|---|---|
| 30 | 0.31 | 1.07 | 3.02 | 2.73 | 1.21 | 1.34 |
| 6 | 2.86 | 2.64 | 3.48 | 3.01 | 1.13 | 2.02 |
| 1.2 | 0.99 | 1.12 | 1.78 | 2.28 | 0.74 | 1.51 |
| 0.24 | 0.78 | 5.73 | 4.38 | 3.36 | 0.55 | 2.36 |
| 0.048 | 1.67 | 3.35 | 3.39 | 0.47 | 3.12 | 3.96 |
| 0 | 0.88 | 2.20 | 4.04 | 3.52 | 2.94 | 2.84 |

The highest inhibition rate is 95.37%, at concentration of 100 µM myricetin and 30 µM cisplatin.
The inhibition rate is 90.42% at 100 µM myricetin only, and the inhibition rate is 66.86% at 30 µM cisplatin only.

MKN45

TABLE 46

monochlorinated myricetin (formula II)+taxol
monochlorinated myricetin (formula II)+taxol-Relatively Cell inhibition of vehicle control(%)

| Tax (nM) LB-1 (µM) | 50 | 20 | 4 | 0.8 | 0.16 | 0 |
|---|---|---|---|---|---|---|
| 1000 | 91.30 | 77.65 | 70.51 | 71.93 | 70.00 | 70.65 |
| 200 | 91.55 | 73.25 | 66.34 | 66.82 | 65.69 | 64.94 |
| 40 | 83.94 | 65.31 | 51.08 | 49.24 | 51.47 | 48.11 |
| 8 | 85.41 | 67.71 | 56.78 | 51.13 | 51.69 | 52.04 |
| 1.6 | 83.06 | 65.82 | 42.82 | 37.83 | 39.51 | 35.15 |
| 0 | 82.41 | 61.24 | -1.47 | -7.46 | -7.30 | -13.69 |
| monochlorinated myricetin (formula II)+taxol-STEDV | | | | | | |
| Tax (nM) LB-1 (µM) | 50 | 20 | 4 | 0.8 | 0.16 | 0 |
| 1000 | 4.61 | 1.94 | 2.58 | 1.58 | 4.01 | 1.36 |
| 200 | 2.60 | 3.59 | 1.64 | 1.00 | 1.10 | 2.45 |
| 40 | 0.92 | 2.90 | 2.98 | 4.10 | 1.84 | 3.40 |
| 8 | 0.66 | 1.91 | 1.58 | 2.42 | 2.78 | 2.98 |
| 1.6 | 0.45 | 1.76 | 5.84 | 2.95 | 3.68 | 1.36 |
| 0 | 1.83 | 0.88 | 3.04 | 1.43 | 1.67 | 4.34 |

The highest inhibition rate is 91.55%, at concentration of 50uM LB-1 and 200 nM taxol.
The inhibition rate is 82.41% at 50 µM LB-1 only, and the inhibition rate is 64.94% at 200 nM taxol only.

TABLE 47

monochlorinated myricetin formula II)+doxorubicin
monochlorinated myricetin (formula II)+doxorubicin-Relatively Cell inhibition of vehicle control(%)

| Dox (nM) LB-1 (µM) | 50 | 20 | 4 | 0.8 | 0.16 | 0 |
|---|---|---|---|---|---|---|
| 10000 | 84.95 | 79.19 | 75.82 | 75.37 | 73.59 | 73.01 |
| 2000 | 96.04 | 95.15 | 95.45 | 95.7 | 95.31 | 95.07 |
| 400 | 90.24 | 76.80 | 68.55 | 71.49 | 67.86 | 65.75 |
| 80 | 86.24 | 66.27 | 32.46 | 28.75 | 32.88 | 28.50 |
| 16 | 85.93 | 64.00 | 10.81 | 8.70 | 4.38 | 2.79 |
| 0 | 86.38 | 66.45 | 11.49 | 2.45 | 2.80 | -1.00 |
| monochlorinated myricetin (formula II)+doxorubicin-STEDV | | | | | | |
| Dox (nM) LB-1 (µM) | 50 | 20 | 4 | 0.8 | 0.16 | 0 |
| 10000 | 5.95 | 0.77 | 3.22 | 7.41 | 5.24 | 6.66 |
| 2000 | 0.54 | 0.50 | 0.59 | 0.16 | 0.22 | 0.32 |
| 400 | 3.03 | 2.79 | 4.10 | 4.93 | 6.65 | 4.99 |
| 80 | 1.76 | 1.22 | 2.99 | 4.31 | 5.42 | 8.33 |
| 16 | 0.47 | 5.61 | 4.62 | 7.94 | 8.56 | 5.68 |
| 0 | 2.14 | 3.58 | 2.88 | 0.99 | 6.46 | 6.43 |

The highest inhibition rate is 96.04%, at concentration of 50 µM LB-1 and 2000 nM doxorubicin.
The inhibition rate is 86.38% at 50 µM LB-1 only, and the inhibition rate is 95.07% at 2000 nM doxorubicin only.

TABLE 48

monochlorinated myricetin formula II+topotecan
monochlorinated myricetin (formula II)+topotecan-Relatively Cell inhibition of vehicle control(%)

| Top (nM) LB-1 (µM) | 50 | 20 | 4 | 0.8 | 0.16 | 0 |
|---|---|---|---|---|---|---|
| 10000 | 90.52 | 90.76 | 90.18 | 90.04 | 90.49 | 89.36 |
| 2000 | 97.87 | 94.83 | 92.14 | 92.27 | 92.63 | 91.55 |
| 400 | 94.63 | 88.09 | 85.07 | 85.55 | 85.37 | 84.58 |
| 80 | 89.54 | 73.63 | 55.54 | 51.24 | 52.47 | 48.98 |
| 16 | 86.15 | 55.77 | 23.22 | 18.54 | 16.50 | 14.86 |
| 0 | 85.97 | 62.53 | 1.64 | -5.68 | -1.11 | -5.33 |
| monochlorinated myricetin (formula II)+topotecan-STEDV | | | | | | |
| Top (nM)LB-1 (µM) | 50 | 20 | 4 | 0.8 | 0.16 | 0 |
| 10000 | 1.07 | 0.19 | 0.34 | 0.65 | 0.61 | 0.58 |
| 2000 | 0.24 | 0.34 | 0.30 | 0.31 | 0.12 | 0.76 |
| 400 | 1.92 | 0.80 | 1.54 | 0.92 | 1.03 | 0.91 |
| 80 | 0.66 | 4.60 | 2.28 | 1.62 | 2.98 | 3.59 |
| 16 | 1.07 | 11.88 | 1.47 | 3.80 | 4.95 | 3.42 |
| 0 | 2.15 | 6.64 | 9.01 | 7.14 | 9.30 | 6.91 |

The highest inhibition rate is 97.87%, at concentration of 50 µM LB-1 and 2000 nM topotecan.
The inhibition rate is 85.97% at 50 µM LB-1 only, and the inhibition rate is 91.55% at 2000 nM topotecan.

TABLE 49

monochlorinated myricetin (formula II)+cisplatin
monochlorinated myricetin (formula II)+cisplatin-Relatively Cell inhibition of vehicle control(%)

| Cis (µM) LB-1 (µM) | 50 | 20 | 4 | 0.8 | 0.16 | 0 |
|---|---|---|---|---|---|---|
| 30 | 94.06 | 90.78 | 83.42 | 82.33 | 82.93 | 81.54 |
| 6 | 87.07 | 51.80 | 30.73 | 31.99 | 34.22 | 33.22 |
| 1.2 | 84.67 | 46.90 | 8.67 | 8.24 | 8.62 | 5.53 |
| 0.24 | 80.67 | 43.94 | 3.13 | 2.82 | 3.58 | -2.82 |
| 0.048 | 80.84 | 66.39 | 16.01 | 9.43 | 11.76 | 7.78 |
| 0 | 76.85 | 55.45 | 19.31 | 8.18 | 12.12 | 3.15 |

monochlorinated myricetin (formula II)+cisplatin-STEDV

| Cis (µM) LB-1 (µM) | 50 | 20 | 4 | 0.8 | 0.16 | 0 |
|---|---|---|---|---|---|---|
| 30 | 0.33 | 0.46 | 0.54 | 1.75 | 0.42 | 1.37 |
| 6 | 2.64 | 11.26 | 5.26 | 2.78 | 1.99 | 1.67 |
| 1.2 | 1.58 | 3.68 | 1.06 | 4.46 | 4.22 | 5.19 |
| 0.24 | 1.35 | 1.59 | 0.44 | 4.56 | 2.57 | 3.90 |
| 0.048 | 3.25 | 4.47 | 2.12 | 2.19 | 2.39 | 4.72 |
| 0 | 9.36 | 8.91 | 4.05 | 0.61 | 4.25 | 6.46 |

The highest inhibition rate is 94.06%, at concentration of 50 µM LB-1 and 30 µM cisplatin.
The inhibition rate is 76.85% at 50 µM LB-1 only, and the inhibition rate is 81.54% at 30 µM cisplatin only.

TABLE 50

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 71.66 | 72.77 | 70.46 | 68.49 | 71.12 | 69.93 |
| 200 | 71.59 | 71.15 | 69.11 | 69.20 | 70.81 | 68.16 |
| 40 | 62.41 | 61.75 | 54.99 | 53.12 | 56.12 | 56.30 |
| 8 | 62.39 | 56.06 | 53.18 | 49.82 | 53.57 | 50.47 |
| 1.6 | 47.95 | 40.76 | 36.39 | 37.22 | 41.1 | 34.79 |
| 0 | 32.89 | 5.06 | -1.75 | 0.13 | 1.30 | -4.34 |
| myricetin+taxol-STEDV | | | | | | |
| Tax (nM) r (µM) | 100 | 20 | 4 | 0.8 | 0.16 | 0 |
| 1000 | 1.79 | 1.56 | 0.49 | 2.29 | 2.76 | 1.69 |
| 200 | 1.52 | 1.69 | 2.26 | 1.32 | 1.93 | 0.22 |
| 40 | 2.68 | 5.12 | 1.99 | 2.96 | 1.35 | 4.28 |
| 8 | 1.46 | 2.38 | 0.48 | 0.59 | 1.69 | 2.14 |
| 1.6 | 0.84 | 3.43 | 0.75 | 2.15 | 0.60 | 2.30 |
| 0 | 4.39 | 3.27 | 2.63 | 4.12 | 5.17 | 6.22 |

The highest inhibition rate is 72.77%, at concentration of 20 µM myricetin and 1000 nM taxol.
The inhibition rate is 5.06% at 20 µM myricetin only, and the inhibition rate is 69.93% at 1000 nM taxol only.

TABLE 51

myricetin+doxorubicin
myricetin+doxorubicin-Relatively Cell inhibition of vehicle control(%)

| Dox (nM) Myr (µM) | 100 | 20 | 4 | 0.8 | 0.16 | 0 |
|---|---|---|---|---|---|---|
| 10000 | 89.81 | 83.19 | 82.66 | 83.05 | 82.75 | 81.99 |
| 2000 | 87.26 | 95.19 | 95.01 | 94.27 | 93.98 | 93.78 |
| 400 | 54.88 | 67.18 | 68.7 | 70.30 | 70.69 | 71.19 |
| 80 | 34.00 | 25.19 | 23.53 | 20.22 | 28.24 | 32.23 |
| 16 | 34.88 | 10.97 | 7.64 | 4.00 | 11.88 | 9.08 |
| 0 | 35.29 | 13.04 | 2.42 | 1.79 | 4.35 | 4.19 |
| myricetin+doxorubicin-STEDV | | | | | | |
| Dox (nM) Myr (µM) | 100 | 20 | 4 | 0.8 | 0.16 | 0 |
| 10000 | 1.68 | 0.88 | 0.29 | 0.58 | 1.33 | 1.27 |
| 2000 | 1.69 | 0.30 | 0.48 | 0.29 | 0.51 | 0.47 |
| 400 | 1.86 | 2.11 | 2.16 | 1.84 | 2.63 | 2.36 |
| 80 | 4.49 | 1.49 | 2.98 | 1.29 | 0.79 | 1.63 |
| 16 | 1.81 | 4.18 | 3.83 | 7.93 | 5.27 | 4.00 |
| 0 | 3.57 | 1.80 | 2.67 | 7.78 | 4.59 | 7.95 |

The highest inhibition rate is 95.19%, at concentration of 20 µM myricetin and 2000 nM doxorubicin.
The inhibition rate is 13.04% at 20 µM myricetin only, and the inhibition rate is 93.78% at 2000 nM doxorubicin only.

TABLE 52

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 93.88 | 92.25 | 91.75 | 91.43 | 91.94 | 91.07 |
| 2000 | 95.03 | 93.94 | 93.57 | 93.15 | 92.71 | 92.29 |
| 400 | 89.07 | 87.16 | 86.96 | 85.26 | 86.20 | 85.99 |
| 80 | 65.14 | 57.03 | 53.58 | 53.62 | 51.25 | 53.47 |
| 16 | 46.69 | 32.00 | 27.97 | 23.36 | 28.50 | 27.49 |
| 0 | 29.93 | 1.70 | -4.89 | -8.02 | -6.95 | -2.58 |
| myricetin+topotecan-STEDV | | | | | | |
| Top (nM) Myr (µM) | 100 | 20 | 4 | 0.8 | 0.16 | 0 |
| 10000 | 0.50 | 0.53 | 0.62 | 0.63 | 0.32 | 0.52 |
| 2000 | 0.20 | 0.50 | 0.25 | 0.47 | 0.15 | 0.08 |
| 400 | 0.68 | 1.03 | 0.79 | 0.93 | 1.54 | 1.12 |
| 80 | 2.62 | 6.82 | 6.69 | 6.17 | 4.41 | 4.53 |
| 16 | 1.51 | 4.12 | 5.99 | 6.87 | 5.18 | 1.45 |
| 0 | 5.09 | 7.28 | 4.73 | 5.82 | 3.99 | 3.58 |

The highest inhibition rate is 95.03%, at concentration of 100 µM myricetin and 2000 nM topotecan.
The inhibition rate is 29.93% at 100 µM myricetin only, and the inhibition rate is 92.29% at 2000 nM topotecan only.

TABLE 53

myricetin+cisplatin
myricetin+cilatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 92.27 | 90.71 | 90.01 | 89.65 | 89.95 | 90.46 |
| 6 | 61.66 | 52.11 | 51.50 | 48.94 | 48.85 | 48.92 |
| 1.2 | 49.05 | 25.30 | 17.94 | 14.54 | 15.05 | 22.76 |
| 0.24 | 41.07 | 9.60 | 5.99 | 4.60 | 5.52 | 4.13 |
| 0.048 | 40.28 | 11.37 | 6.96 | 0.53 | 6.46 | 5.10 |
| 0 | 44.00 | 14.42 | 6.78 | 5.96 | 7.46 | 2.15 |
| myricetin+cisplatin-STEDV | | | | | | |
| Cis (µM) Myr (µM) | 100 | 20 | 4 | 0.8 | 0.16 | 0 |
| 30 | 2.05 | 3.30 | 3.63 | 3.48 | 3.93 | 2.73 |
| 6 | 6.75 | 8.31 | 7.18 | 9.06 | 8.03 | 8.58 |
| 1.2 | 2.38 | 8.27 | 4.66 | 1.12 | 2.99 | 5.76 |
| 0.24 | 6.43 | 2.47 | 4.70 | 2.16 | 3.05 | 2.62 |
| 0.048 | 3.97 | 2.77 | 4.50 | 1.83 | 6.32 | 2.11 |
| 0 | 6.25 | 1.78 | 2.36 | 3.13 | 0.54 | 3.47 |

The highest inhibition rate is 92.27%, at concentration of 100 µM myricetin and 30 µM cisplatin.
The inhibition rate is 44% at 100 µM myricetin only, and the inhibition rate is 90.46% at 30 µM cisplatin only.

786-0

TABLE 54

monochlorinated myricetin (formula II)+taxol
monochlorinated myricetin (formula II)+taxol-Relatively Cell inhibition of vehicle control(%)

| Tax (nM) LB-1 (µM) | 50 | 20 | 4 | 0.8 | 0.16 | 0 |
|---|---|---|---|---|---|---|
| 1000 | 93.76 | 73.19 | 73.50 | 72.25 | 72.84 | 73.05 |
| 200 | 93.45 | 73.61 | 68.29 | 69.03 | 69.54 | 69.53 |
| 40 | 92.86 | 67.10 | 42.60 | 42.79 | 41.77 | 43.18 |
| 8 | 91.99 | 51.84 | 17.41 | 12.79 | 14.99 | 12.57 |
| 1.6 | 91.45 | 47.50 | 7.29 | 3.67 | 3.25 | 3.15 |
| 0 | 90.16 | 35.62 | 1.19 | -0.28 | 0.46 | 0.14 |
| monochlorinated myricetin (formula II)+taxol-STEDV | | | | | | |
| Tax (nM) LB-1 (µM) | 50 | 20 | 4 | 0.8 | 0.16 | 0 |
| 1000 | 0.62 | 2.04 | 1.46 | 1.74 | 1.78 | 1.84 |
| 200 | 0.32 | 1.73 | 1.49 | 0.78 | 2.21 | 2.25 |
| 40 | 0.27 | 1.28 | 4.56 | 4.54 | 4.14 | 4.41 |
| 8 | 0.68 | 1.98 | 4.65 | 5.18 | 5.58 | 4.25 |
| 1.6 | 0.58 | 1.78 | 1.59 | 2.29 | 0.98 | 0.98 |
| 0 | 1.32 | 3.07 | 3.42 | 3.66 | 1.06 | 2.17 |

The highest inhibition rate is 93.76%, at concentration of 50 µM LB-1 and 1000 nM taxol.
The inhibition rate is 90.16% at 50 µM LB-1 only, and the inhibition rate is 73.05% at 1000 nM taxol only.

TABLE 55

monochlorinated myricetin (formula II)+doxorubicin
monochlorinated myricetin (formula II)+doxorubicin-Relatively Cell inhibition of vehicle control(%)

| Dox (nM) LB-1 (µM) | 50 | 20 | 4 | 0.8 | 0.16 | 0 |
|---|---|---|---|---|---|---|
| 10000 | 83.86 | 78.19 | 80.69 | 80.52 | 80.18 | 79.94 |
| 2000 | 88.60 | 83.70 | 79.59 | 78.53 | 79.45 | 77.77 |
| 400 | 92.11 | 78.73 | 64.05 | 62.34 | 63.50 | 62.24 |
| 80 | 91.39 | 57.59 | 27.57 | 24.01 | 26.11 | 24.26 |
| 16 | 91.75 | 48.71 | 8.72 | 5.01 | 7.21 | 3.98 |
| 0 | 91.60 | 40.28 | 5.86 | 1.50 | 4.73 | 1.95 |
| monochlorinated myricetin (formula II)+doxorubicin-STEDV | | | | | | |
| Dox (nM) LB-1 (µM) | 50 | 20 | 4 | 0.8 | 0.16 | 0 |
| 10000 | 0.53 | 0.86 | 1.03 | 0.34 | 1.31 | 1.41 |
| 2000 | 0.29 | 0.52 | 0.57 | 0.96 | 0.12 | 0.65 |
| 400 | 0.28 | 0.60 | 0.70 | 0.58 | 1.65 | 1.25 |
| 80 | 0.34 | 2.67 | 1.30 | 2.20 | 0.93 | 0.14 |
| 16 | 0.45 | 4.67 | 0.92 | 1.41 | 0.97 | 1.73 |
| 0 | 0.71 | 4.44 | 2.09 | 1.03 | 0.73 | 1.49 |

The highest inhibition rate is 92.11%, at concentration of 50 µM LB-1 and 400 nM doxorubicin.
The inhibition rate is 91.6% at 50 µM LB-1 only, and the inhibition rate is 62.24% at 400 nM doxorubicin only.

TABLE 56

monochlorinated myricetin (formula II)+topotecan
monochlorinated myricetin (formula II)+topotecan-Relatively Cell inhibition of vehicle control(%)

| Top (nM) LB-1 (µM) | 50 | 20 | 4 | 0.8 | 0.16 | 0 |
|---|---|---|---|---|---|---|
| 10000 | 92.65 | 95.31 | 94.41 | 94.23 | 94.70 | 93.59 |
| 2000 | 96.45 | 98.31 | 91.40 | 91.67 | 92.17 | 91.65 |
| 400 | 95.20 | 92.23 | 84.26 | 83.69 | 83.31 | 83.15 |
| 80 | 92.16 | 76.01 | 70.03 | 69.86 | 68.03 | 68.14 |
| 16 | 91.56 | 60.53 | 9.89 | 6.05 | 6.51 | 2.61 |
| 0 | 91.54 | 39.97 | 4.62 | 1.93 | 0.88 | -0.37 |

monochlorinated myricetin (formula II)+topotecan-STEDV

| Top (nM) LB-1(µM) | 50 | 20 | 4 | 0.8 | 0.16 | 0 |
|---|---|---|---|---|---|---|
| 10000 | 0.96 | 0.64 | 1.21 | 0.71 | 0.61 | 1.02 |
| 2000 | 0.48 | 0.15 | 0.82 | 0.98 | 0.57 | 0.97 |
| 400 | 0.65 | 0.76 | 1.25 | 0.78 | 0.91 | 0.41 |
| 80 | 1.19 | 2.21 | 1.06 | 1.08 | 1.46 | 0.90 |
| 16 | 1.00 | 3.44 | 4.45 | 4.48 | 1.54 | 2.30 |
| 0 | 1.21 | 2.36 | 2.29 | 3.33 | 2.88 | 1.43 |

The highest inhibition rate is 98.31%, at concentration of 20 µM LB-1 and 2000 nM topotecan.
The inhibition rate is 39.97% at 50 µM LB-1 only, and the inhibition rate is 91.65% at 2000 nM topotecan only.

TABLE 57

monochlorinated myricetin (formula II)+cisplatin
monochlorinated myricetin (formula II)+cisplatin-Relatively Cell inhibition of vehicle control(%)

| Cis (µM) LB-1 (µM) | 50 | 20 | 4 | 0.8 | 0.16 | 0 |
|---|---|---|---|---|---|---|
| 30 | 88.34 | 91.54 | 90.69 | 90.22 | 88.94 | 90.04 |
| 6 | 95.28 | 73.07 | 60.15 | 54.08 | 50.29 | 60.32 |
| 1.2 | 91.66 | 52.77 | 8.54 | 3.43 | 3.59 | 5.31 |
| 0.24 | 90.81 | 37.58 | 3.21 | -2.21 | -0.98 | -1.54 |
| 0.048 | 90.87 | 46.20 | 3.28 | -0.69 | 1.86 | 0.45 |
| 0 | 91.22 | 39.02 | 5.46 | 1.24 | 3.97 | 2.79 |
| monochlorinated myricetin (formula II)+ cisplatin-STEDV | | | | | | |
| Cis (µM) LB-1 (µM) | 50 | 20 | 4 | 0.8 | 0.16 | 0 |
| 30 | 1.31 | 1.38 | 0.81 | 0.51 | 1.36 | 1.40 |
| 6 | 0.29 | 1.26 | 1.57 | 1.93 | 1.23 | 1.67 |
| 1.2 | 0.21 | 3.20 | 1.08 | 1.07 | 2.18 | 1.56 |
| 0.24 | 0.37 | 4.86 | 1.35 | 1.72 | 0.61 | 1.36 |
| 0.048 | 0.07 | 4.39 | 1.68 | 0.69 | 2.30 | 1.04 |
| 0 | 0.21 | 2.72 | 0.85 | 0.70 | 1.72 | 1.32 |

The highest inhibition rate is 95.28%, at concentration of 50 µM LB-1 and 6 µM cisplatin.
The inhibition rate is 91.22% at 50 µM LB-1 only, and the inhibition rate is 60.32% at 6 µM cisplatin only.

TABLE 58

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 82.02 | 75.52 | 73.77 | 73.43 | 73.70 | 74.06 |
| 200 | 77.31 | 72.03 | 70.63 | 69.59 | 69.71 | 70.00 |
| 40 | 58.14 | 48.01 | 43.45 | 41.36 | 42.66 | 42.62 |
| 8 | 50.18 | 21.17 | 14.87 | 11.13 | 12.79 | 12.20 |
| 1.6 | 43.87 | 10.16 | 3.82 | 2.06 | 1.53 | 2.78 |
| 0 | 41.97 | 9.84 | 2.60 | 0.33 | 1.90 | 2.73 |
| myricetin+taxol-STEDV | | | | | | |
| Tax (nM) Myr (µM) | 100 | 20 | 4 | 0.8 | 0.16 | 0 |
| 1000 | 1.03 | 0.68 | 0.92 | 1.11 | 0.91 | 0.52 |
| 200 | 1.02 | 1.30 | 0.66 | 0.97 | 1.03 | 1.30 |
| 40 | 1.17 | 1.72 | 1.90 | 0.46 | 0.99 | 0.96 |
| 8 | 2.23 | 1.37 | 1.62 | 2.05 | 0.51 | 2.02 |
| 1.6 | 1.37 | 1.79 | 1.36 | 2.03 | 1.55 | 1.40 |
| 0 | 0.76 | 2.15 | 2.88 | 1.50 | 2.90 | 1.88 |

The highest inhibition rate is 82.02%, at concentration of 100 µM myricetin and 1000 nM taxol.
The inhibition rate is 41.97% at 100 µM myricetin only, and the inhibition rate is 74.06% at 1000 nM taxol only.

TABLE 59

myricetin+doxorubicin
myricetin+doxorubicin-Relatively Cell inhibition of vehicle control(%)

| Dox (nM) Myr (µM) | 100 | 20 | 4 | 0.8 | 0.16 | 0 |
|---|---|---|---|---|---|---|
| 10000 | 85.17 | 83.98 | 83.59 | 82.88 | 84.02 | 83.82 |
| 2000 | 79.52 | 78.52 | 77.08 | 76.69 | 76.21 | 76.23 |
| 400 | 48.75 | 51.56 | 61.52 | 64.39 | 64.71 | 64.93 |
| 80 | 44.4 | 29.21 | 22.14 | 23.82 | 22.53 | 24.00 |
| 16 | 36.64 | 8.80 | 7.03 | 2.87 | 2.30 | 4.40 |
| 0 | 43.15 | 11.20 | 4.47 | 0.86 | 2.43 | 2.33 |

myricetin+doxorubicin-STEDV

| Dox (nM) Myr (µM) | 100 | 20 | 4 | 0.8 | 0.16 | 0 |
|---|---|---|---|---|---|---|
| 10000 | 0.32 | 0.34 | 0.45 | 0.16 | 0.99 | 0.49 |
| 2000 | 1.10 | 1.14 | 0.67 | 0.49 | 0.85 | 1.03 |
| 400 | 0.42 | 4.02 | 2.46 | 1.96 | 1.48 | 1.17 |
| 80 | 0.90 | 3.36 | 5.80 | 0.69 | 4.21 | 1.94 |
| 16 | 0.50 | 0.63 | 3.59 | 0.85 | 3.52 | 0.86 |
| 0 | 0.37 | 2.69 | 1.06 | 3.34 | 1.60 | 1.41 |

The highest inhibition rate is 85.17%, at concentration of 100 µM myricetin and 10000 nM doxorubicin.
The inhibition rate is 43.15% at 100 µM myricetin only, and the inhibition rate is 83.82% at 10000 nM doxorubicin only.

TABLE 60

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 96.42 | 95.00 | 94.33 | 94.30 | 94.28 | 93.86 |
| 2000 | 94.53 | 92.68 | 92.23 | 92.40 | 92.13 | 91.68 |
| 400 | 86.75 | 82.94 | 81.71 | 82.15 | 82.16 | 82.54 |
| 80 | 76.51 | 69.40 | 66.72 | 67.14 | 67.94 | 68.07 |
| 16 | 48.12 | 13.67 | 4.53 | 4.93 | 5.58 | 4.00 |
| 0 | 44.80 | 10.39 | 4.03 | 0.12 | 1.25 | 3.11 |
| myricetin+topotecan-STEDV | | | | | | |
| Top (nM) Myr (µM) | 100 | 20 | 4 | 0.8 | 0.16 | 0 |
| 10000 | 0.43 | 0.47 | 0.68 | 0.67 | 0.50 | 0.56 |
| 2000 | 0.60 | 1.00 | 0.25 | 0.92 | 1.07 | 0.87 |
| 400 | 0.91 | 1.75 | 1.44 | 0.43 | 0.93 | 1.30 |
| 80 | 1.31 | 1.31 | 2.54 | 1.75 | 1.15 | 1.59 |
| 16 | 5.26 | 1.97 | 2.98 | 0.82 | 3.63 | 2.15 |
| 0 | 2.14 | 2.87 | 1.43 | 1.84 | 0.33 | 3.87 |

The highest inhibition rate is 96.42%, at concentration of 100 µM myricetin and 10000 nM topotecan.
The inhibition rate is 44.8% at 100 µM myricetin only, and the inhibition rate is 93.86% at 10000 nM topotecan only.

TABLE 61

myricetin+cisplatin
myricetin+cilatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 93.88 | 90.13 | 88.20 | 87.66 | 87.90 | 86.78 |
| 6 | 75.75 | 68.76 | 67.04 | 67.44 | 67.04 | 67.62 |
| 1.2 | 47.85 | 19.05 | 12.44 | 11.10 | 10.75 | 10.33 |
| 0.24 | 38.24 | 7.95 | 1.62 | -2.28 | -2.11 | -0.68 |
| 0.048 | 40.54 | 8.91 | 1.04 | -1.06 | 0.89 | -1.09 |
| 0 | 43.41 | 10.78 | 2.54 | 0.79 | -0.49 | 0.79 |
| myricetin+cisplatin-STEDV | | | | | | |
| Cis (µM) Myr (µM) | 100 | 20 | 4 | 0.8 | 0.16 | 0 |
| 30 | 0.46 | 0.61 | 0.85 | 0.55 | 0.82 | 0.20 |
| 6 | 0.42 | 2.51 | 0.81 | 0.41 | 1.31 | 1.54 |
| 1.2 | 0.82 | 1.78 | 1.91 | 1.53 | 0.99 | 0.71 |
| 0.24 | 0.97 | 1.13 | 1.86 | 1.83 | 1.86 | 1.08 |
| 0.048 | 3.17 | 1.52 | 0.20 | 0.77 | 0.20 | 1.01 |
| 0 | 2.07 | 2.95 | 0.54 | 2.01 | 1.98 | 1.41 |

The highest inhibition rate is 93.88%, at concentration of 100 µM myricetin and 30 µM cisplatin.
The inhibition rate is 43.41% at 100 µM myricetin only, and the inhibition rate is 86.78% at 30 µM cisplatin only.

### SK-Hep-1

TABLE 62

monochlorinated myricetin (formula II)+taxol
monochlorinated myricetin (formula II)+taxol-Relatively Cell inhibition of vehicle control(%)

| Tax (nM) LB-1 (µM) | 100 | 20 | 4 | 0.8 | 0.16 | 0 |
|---|---|---|---|---|---|---|
| 1000 | 80.08 | 89.75 | 82.05 | 76.45 | 78.28 | 78.05 |
| 200 | 76.88 | 84.24 | 74.69 | 63.88 | 64.39 | 68.83 |
| 40 | 76.78 | 80.41 | 61.62 | 51.53 | 54.78 | 59.47 |
| 8 | 75.03 | 71.56 | 38.09 | 21.87 | 25.80 | 30.60 |
| 1.6 | 65.72 | 67.22 | 10.73 | -29.09 | -21.35 | 0.78 |
| 0 | 74.81 | 69.25 | 22.85 | -25.66 | -25.86 | -9.92 |
| monochlorinated myricetin (formula II)+taxol-STEDV | | | | | | |
| Tax (nM)LB-1 (µM) | 100 | 20 | 4 | 0.8 | 0.16 | 0 |
| 1000 | 1.93 | 0.64 | 1.26 | 0.86 | 1.91 | 1.00 |
| 200 | 3.34 | 1.45 | 2.54 | 0.25 | 2.76 | 3.21 |
| 40 | 1.78 | 2.18 | 2.55 | 1.09 | 1.69 | 3.85 |
| 8 | 2.88 | 0.45 | 1.78 | 7.26 | 6.39 | 9.64 |
| 1.6 | 18.18 | 3.10 | 6.40 | 10.15 | 7.47 | 7.75 |
| 0 | 2.03 | 7.83 | 13.40 | 7.80 | 14.41 | 22.49 |

The highest inhibition rate is 89.75%, at concentration of 20 µM LB-1 and 1000 nM taxol.
The inhibition rate is 69.25% at 20 µM LB-1 only, and the inhibition rate is 78.05% at 1000 nM taxol only.

TABLE 63

monochlorinated myricetin (formula II)+doxorubicin
monochlorinated myricetin (formula II)+doxorubicin-Relatively Cell inhibition of vehicle control(%)

| Dox (nM) LB-1 (µM) | 100 | 20 | 4 | 0.8 | 0.16 | 0 |
|---|---|---|---|---|---|---|
| 10000 | 99.85 | 99.69 | 99.65 | 99.65 | 99.66 | 99.41 |
| 2000 | 99.64 | 98.64 | 98.57 | 98.42 | 98.71 | 98.59 |
| 400 | 82.33 | 60.69 | 60.88 | 61.18 | 64.5 | 66.23 |
| 80 | 77.88 | 88.39 | 36.63 | 20.33 | 23.19 | 31.30 |
| 16 | 76.98 | 80.14 | 19.83 | -2.46 | 2.73 | 4.48 |
| 0 | 76.96 | 44.83 | 8.52 | -20.45 | -11.6 | -0.34 |
| monochlorinated myricetin (formula II)+doxorubicin-STEDV | | | | | | |
| Dox (nM) LB-1 (µM) | 100 | 20 | 4 | 0.8 | 0.16 | 0 |
| 10000 | 0.03 | 0.03 | 0.12 | 0.07 | 0.12 | 0.09 |
| 2000 | 0.08 | 0.17 | 0.34 | 0.23 | 0.14 | 0.27 |
| 400 | 3.20 | 2.08 | 8.38 | 8.93 | 8.99 | 5.67 |
| 80 | 2.02 | 0.65 | 4.59 | 7.49 | 2.66 | 8.01 |
| 16 | 5.05 | 4.43 | 4.61 | 11.83 | 13.53 | 10.25 |
| 0 | 2.49 | 18.71 | 14.50 | 4.03 | 13.00 | 8.28 |

The highest inhibition rate is 99.85%, at concentration of 100 µM LB-1 and 10000 nM doxorubicin.
The inhibition rate is 76.96% at 100 µM LB-1 only, and the inhibition rate is 99.41% at 10000 nM doxorubicin only.

TABLE 64

monochlorinated myricetin (formula II)+topotecan
monochlorinated myricetin (formula II)+topotecan-Relatively Cell inhibition of vehicle control(%)

| Top (nM) LB-1 (µM) | 100 | 20 | 4 | 08 | 0.16 | 0 |
|---|---|---|---|---|---|---|
| 10000 | 99.81 | 98.91 | 98.85 | 98.61 | 098.47 | 98.59 |
| 2000 | 99.22 | 81.34 | 69.44 | 66.03 | 69.54 | 70.32 |
| 400 | 88.52 | 78.25 | 31.37 | 23.89 | 26.68 | 35.39 |
| 80 | 76.42 | 87.70 | 30.85 | -7.11 | -2.25 | 9.52 |
| 16 | 74.97 | 83.92 | 19.89 | -32.82 | -26.72 | -0.38 |
| 0 | 77.16 | 70.28 | 34.75 | -26.96 | -17.54 | -1.50 |
| monochlorinated myricetin (formula II)+topotecan-STEDV | | | | | | |
| Top (nM) LB-1 (µM) | 100 | 20 | 4 | 08 | 0.16 | 0 |
| 10000 | 0.04 | 0.38 | 0.33 | 0.37 | 0.25 | 0.54 |
| 2000 | 0.20 | 0.94 | 3.58 | 3.35 | 3.12 | 2.96 |
| 400 | 0.61 | 3.88 | 3.02 | 2.69 | 5.38 | 7.19 |
| 80 | 1.97 | 0.36 | 6.12 | 12.35 | 4.59 | 0.77 |
| 16 | 1.76 | 0.98 | 7.78 | 8.44 | 14.72 | 5.80 |
| 0 | 3.58 | 1.11 | 1.64 | 12.57 | 3.41 | 5.57 |

The highest inhibition rate is 99.81%, at concentration of 100uM LB-1 and 10000 nM topotecan.
The inhibition rate is 77.16% at 100 µM LB-1 only, and the inhibition rate is 98.59% at 1000 nM topotecan only.

TABLE 65

monochlorinated myricetin (formula II)+cisplatin
monochlorinated myricetin (formula II)+cisplatin-Relatively Cell inhibition of vehicle control(%)

| Cis (µM) LB-1 (µM) | 100 | 20 | 4 | 0.8 | 0.16 | 0 |
|---|---|---|---|---|---|---|
| 30 | 95.36 | 91.22 | 78.35 | 63.42 | 65.37 | 70.68 |
| 6 | 69.44 | 89.53 | 63.19 | 20.24 | 11.94 | 29.88 |
| 1.2 | 55.82 | 81.13 | 33.88 | -17.16 | -22.03 | -10.98 |
| 0.24 | 68.54 | 72.65 | 18.48 | -36.76 | -29.57 | -16.48 |
| 0.048 | 63.60 | 52.67 | 20.22 | -30.69 | -20.68 | -8.71 |
| 0 | 61.33 | 53.83 | 38.34 | -18.07 | -5.85 | -0.44 |

monochlorinated myricetin (formula II)+cisplatin-STEDV

| Cis (µM) LB-1 (µM) | 100 | 20 | 4 | 0.8 | 0.16 | 0 |
|---|---|---|---|---|---|---|
| 30 | 0.95 | 0.75 | 2.65 | 1.90 | 3.77 | 2.59 |
| 6 | 9.29 | 1.51 | 2.00 | 9.78 | 7.18 | 7.66 |
| 1.2 | 9.80 | 3.34 | 2.90 | 6.33 | 8.32 | 1.84 |
| 0.24 | 3.25 | 3.10 | 16.17 | 12.67 | 14.69 | 3.69 |
| 0.048 | 10.66 | 29.30 | 9.29 | 5.22 | 6.97 | 5.86 |
| 0 | 10.53 | 24.04 | 4.90 | 3.00 | 4.19 | 4.61 |

The highest inhibition rate is 95.36%, at concentration of 100 µM LB-1 and 30 µM cisplatin.
The inhibition rate is 61.33% at 100 µM LB-1 only, and the inhibition rate is 70.68% at 30 µM cisplatin only.

TABLE 66

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 99.67 | 95.35 | 85.14 | 79.43 | 78.82 | 78.84 |
| 200 | 99.57 | 94.67 | 76.36 | 69.31 | 68.04 | 65.15 |
| 40 | 99.45 | 94.39 | 69.52 | 61.33 | 62.24 | 62.41 |
| 8 | 99.36 | 91.48 | 45.46 | 38.41 | 31.29 | 35.99 |
| 1.6 | 99.32 | 90.39 | 14.93 | -0.74 | -3.89 | 0.58 |
| 0 | 99.57 | 92.78 | 22.81 | -6.66 | 2.96 | 4.46 |

myricetin+taxol-STEDV

| Tax (nM Myr (µM) | 100 | 20 | 4 | 0.8 | 0.16 | 0 |
|---|---|---|---|---|---|---|
| 1000 | 0.07 | 1.02 | 1.36 | 2.36 | 2.18 | 0.83 |
| 200 | 0.09 | 1.58 | 2.18 | 1.95 | 2.00 | 3.65 |
| 40 | 0.20 | 1.05 | 1.28 | 2.06 | 1.17 | 1.37 |
| 8 | 0.10 | 1.71 | 4.30 | 4.06 | 1.93 | 1.28 |
| 1.6 | 0.10 | 2.94 | 7.37 | 2.78 | 5.67 | 4.49 |
| 0 | 0.03 | 2.10 | 6.50 | 4.62 | 11.14 | 6.93 |

The highest inhibitory rate is 99.67%, at concentration of 100uM myricetin and 1000 nM taxol.
The inhibition rate is 99.57% at 100 µM myricetin only, and the inhibition rate is 78.84% at 1000 nM taxol only.

TABLE 67

myricetin+doxorubicin
myricetin+doxorubicin-Relatively Cell inhibition of vehicle control(%)

| Dox (nM) Myr (µM) | 100 | 20 | 4 | 0.8 | 0.16 | 0 |
|---|---|---|---|---|---|---|
| 10000 | 99.98 | 99.94 | 99.79 | 99.73 | 99.71 | 99.43 |
| 2000 | 99.77 | 99.81 | 98.74 | 98.38 | 98.30 | 98.50 |
| 400 | 99.39 | 89.93 | 61.64 | 64.19 | 65.51 | 68.47 |
| 80 | 99.16 | 94.08 | 39.45 | 22.13 | 16.71 | 25.49 |
| 16 | 99.28 | 94.15 | 35.21 | -5.94 | -4.00 | 6.89 |
| 0 | 99.41 | 93.53 | 32.28 | -9.74 | 1.44 | 3.21 |

myricetin+doxorubicin-STEDV

| Dox (nM) Myr (µM) | 100 | 20 | 4 | 0.8 | 0.16 | 0 |
|---|---|---|---|---|---|---|
| 10000 | 0.00 | 0.01 | 0.04 | 0.03 | 0.03 | 0.05 |
| 2000 | 0.24 | 0.04 | 0.05 | 0.50 | 0.28 | 0.26 |
| 400 | 0.14 | 1.29 | 1.05 | 2.39 | 3.41 | 3.35 |
| 80 | 0.17 | 0.62 | 2.18 | 1.37 | 4.19 | 1.40 |
| 16 | 0.12 | 1.24 | 7.18 | 3.09 | 4.36 | 7.57 |
| 0 | 0.10 | 1.07 | 9.61 | 3.08 | 2.44 | 9.88 |

The highest inhibitory rate is 99.98%, at concentration of 100 µM myricetin and 10000 nM doxorubicin.
The inhibition rate is 99.41 % at 100 µM myricetin only, and the inhibition rate is 99.43% at 10000 nM doxorubicin only.

TABLE 68

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 99.95 | 99.69 | 99.13 | 98.91 | 98.99 | 99.07 |
| 2000 | 99.80 | 96.44 | 75.51 | 75.45 | 73.89 | 74.15 |
| 400 | 99.59 | 93.92 | 42.04 | 40.50 | 36.69 | 37.68 |
| 80 | 99.40 | 94.95 | 30.79 | 13.88 | 12.40 | 16.02 |
| 16 | 99.37 | 94.34 | 21.31 | -9.83 | -13.15 | -4.24 |
| 0 | 99.43 | 93.22 | 16.77 | -19.33 | -14.36 | -6.03 |

myricetin+topotecan-STEDV

| Top (nM) Myr (µM) | 100 | 20 | 4 | 0.8 | 0.16 | 0 |
|---|---|---|---|---|---|---|
| 10000 | 0.03 | 0.05 | 0.12 | 0.14 | 0.11 | 0.11 |
| 2000 | 0.02 | 0.54 | 1.13 | 1.01 | 1.44 | 3.55 |
| 400 | 0.08 | 0.95 | 1.50 | 0.35 | 2.88 | 1.40 |
| 80 | 0.14 | 0.91 | 0.93 | 1.90 | 7.59 | 1.65 |
| 16 | 0.26 | 0.17 | 5.96 | 1.35 | 8.60 | 1.01 |
| 0 | 0.10 | 0.67 | 3.10 | 13.90 | 5.55 | 9.29 |

The highest inhibitory rate is 99.95%, at concentration of 100uM myricetin and 10000 nM topotecan.
The inhibition rate is 99.43% at 100 µM myricetin only, and the inhibition rate is 99.07% at 10000 nM topotecan only.

TABLE 69

myricetin+cisplatin
myricetin+cilatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 99.77 | 96.21 | 82.16 | 76.02 | 76.18 | 76.71 |
| 6 | 99.54 | 95.05 | 59.41 | 29.01 | 22.51 | 29.55 |
| 1.2 | 99.29 | 93.56 | 29.27 | -3.42 | -7.83 | -2.56 |
| 0.24 | 99.48 | 92.88 | 13.54 | -18.41 | -24.18 | -10.23 |
| 0.048 | 99.59 | 92.53 | 15.52 | -23.87 | -28.59 | -7.69 |
| 0 | 99.56 | 92.37 | 20.22 | -11.53 | -4.73 | 7.18 |
| myricetin+cisplatin-STEDV | | | | | | |
| Cis (µM) Myr (µM) | 100 | 20 | 4 | 0.8 | 0.16 | 0 |
| 30 | 0.08 | 0.86 | 1.58 | 2.33 | 3.19 | 1.64 |
| 6 | 0.04 | 0.88 | 5.41 | 7.54 | 8.37 | 3.20 |
| 1.2 | 0.19 | 0.10 | 3.95 | 7.75 | 5.99 | 1.02 |
| 0.24 | 0.13 | 1.26 | 1.41 | 8.95 | 6.34 | 5.04 |
| 0.048 | 0.17 | 0.92 | 9.93 | 4.69 | 4.02 | 8.08 |
| 0 | 0.14 | 0.45 | 9.10 | 6.05 | 6.01 | 15.97 |

The highest inhibition rate is 99.77%, at concentration of 100 µM myricetin and 30 µM cisplatin.
The inhibition rate is 99.56% at 100 µM myricetin only, and the inhibition rate is 76.71% at 30 µM cisplatin only.

### Capan-1

TABLE 70

monochlorinated myricetin (formula II)+taxol
monochlorinated myricetin (formula II)+taxol-Relatively Cell inhibition of vehicle control(%)

| Tax (nM) LB-1 (µM) | 50 | 20 | 4 | 0.8 | 0.16 | 0 |
|---|---|---|---|---|---|---|
| 1000 | 78.30 | 68.96 | 68.21 | 66.53 | 66.34 | 67.21 |
| 200 | 76.08 | 69.30 | 67.17 | 67.08 | 64.45 | 65.39 |
| 40 | 76.08 | 65.35 | 66.28 | 66.28 | 66.43 | 65.78 |
| 8 | 73.43 | 55.72 | 59.22 | 53.97 | 51.56 | 54.34 |
| 1.6 | 71.78 | 35.36 | 18.83 | 16.30 | 16.10 | 17.22 |
| 0 | 71.21 | 33.22 | 12.49 | 4.19 | 5.37 | 10.77 |

monochlorinated myricetin (formula II)+taxol-STEDV

| Tax (nM) LB-1 (µM) | 50 | 20 | 4 | 0.8 | 0.16 | 0 |
|---|---|---|---|---|---|---|
| 1000 | 1.10 | 1.26 | 2.10 | 0.06 | 0.43 | 1.92 |
| 200 | 0.85 | 1.14 | 2.61 | 0.61 | 2.68 | 1.35 |
| 40 | 1.90 | 2.71 | 2.10 | 2.05 | 1.10 | 0.37 |
| 8 | 0.88 | 4.52 | 2.48 | 1.62 | 2.36 | 2.59 |
| 1.6 | 4.74 | 6.19 | 2.07 | 8.88 | 1.05 | 3.50 |
| 0 | 1.27 | 3.01 | 1.99 | 6.72 | 4.97 | 9.99 |

The highest inhibition rate is 78.3%, at concentration of 50uM LB-1 and 1000 nM taxol.
The inhibition rate is 71.21% at 50 µM LB-1 only, and the inhibition rate is 67.21 % at 1000 nM taxol only.

TABLE 71

monochlorinated myricetin (formula II)+doxorubicin
monochlorinated myricetin (formula II)+doxorubicin-Relatively Cell inhibition of vehicle control(%)

| Dox (nM) LB-1 (µM) | 50 | 20 | 4 | 0.8 | 0.16 | 0 |
|---|---|---|---|---|---|---|
| 10000 | 81.34 | 76.01 | 75.74 | 74.09 | 75.07 | 74.86 |
| 2000 | 90.49 | 88.32 | 87.53 | 87.66 | 86.92 | 86.89 |
| 400 | 79.60 | 70.52 | 67.14 | 67.59 | 66.67 | 68.03 |
| 80 | 72.56 | 48.79 | 45.20 | 42.25 | 41.31 | 44.79 |
| 16 | 67.88 | 37.96 | 22.17 | 19.34 | 16.26 | 19.23 |
| 0 | 72.02 | 34.36 | 13.80 | 6.66 | 9.27 | 12.44 |

Monochlorinated myricetin (formula II)+Doxorubicin-STEDV

| Dox (nM) LB-1 (µM) | 50 | 20 | 4 | 0.8 | 0.16 | 0 |
|---|---|---|---|---|---|---|
| 10000 | 0.39 | 0.63 | 0.27 | 0.11 | 1.96 | 1.60 |
| 2000 | 0.30 | 1.05 | 0.61 | 1.18 | 0.87 | 0.39 |
| 400 | 0.29 | 1.58 | 3.50 | 1.96 | 2.71 | 2.63 |
| 80 | 1.22 | 4.98 | 2.18 | 2.94 | 5.03 | 1.02 |
| 16 | 5.95 | 2.34 | 3.98 | 1.25 | 5.31 | 3.52 |
| 0 | 2.38 | 2.74 | 1.41 | 1.64 | 2.29 | 3.56 |

The highest inhibition rate is 90.49%, at concentration of 50 µM LB-1 and 2000 nM doxorubicin.
The inhibition rate is. 72.02% at 50 µM LB-1 only, and the inhibition rate is 86.89% at 2000 nM doxorubicin only.

TABLE 72

monochlorinated myricetin formula II+topotecan
monochlorinated myricetin (formula II)+topotecan-Relatively Cell inhibition of vehicle control(%)

| Top (nM) LB-1 (µM) | 50 | 20 | 4 | 0.8 | 0.16 | 0 |
|---|---|---|---|---|---|---|
| 10000 | 81.28 | 79.17 | 77.09 | 78.21 | 76.62 | 78.14 |
| 2000 | 93.49 | 88.81 | 88.89 | 88.07 | 87.63 | 88.36 |
| 400 | 82.89 | 74.35 | 75.41 | 75.43 | 74.09 | 74.29 |
| 80 | 75.99 | 59.4 | 55.89 | 57.10 | 54.83 | 53.87 |
| 16 | 74.30 | 42.23 | 25.16 | 25.27 | 21.13 | 21.02 |
| 0 | 70.43 | 34.63 | 11.99 | 14.98 | 4.05 | 12.23 |

monochlorinated myricetin (formula II)+topotecan-STEDV

| Top (nM) LB-1 (µM) | 50 | 20 | 4 | 0.8 | 0.16 | 0 |
|---|---|---|---|---|---|---|
| 10000 | 0.50 | 1.33 | 0.37 | 0.29 | 1.58 | 0.77 |
| 2000 | 1.18 | 0.76 | 0.31 | 0.69 | 0.50 | 0.28 |
| 400 | 0.14 | 0.79 | 0.96 | 1.94 | 1.26 | 1.87 |
| 80 | 1.36 | 1.53 | 1.34 | 1.44 | 3.12 | 0.41 |
| 16 | 2.82 | 1.86 | 8.00 | 6.23 | 6.30 | 7.21 |
| 0 | 1.89 | 5.09 | 4.38 | 4.89 | 2.14 | 4.12 |

The highest inhibition rate is 93.49%, at concentration of 50uM LB-1 and 2000 nM topotecan.
The inhibition rate is 70.43% at 50 µM LB-1 only, and the inhibition rate is 88.36% at 2000 nM topotecan only.

TABLE 73

monochlorinated myricetin (formula II)+cisplatin
monochlorinated myricetin (formula II)+cisplatin-Relatively Cell inhibition of vehicle control(%)

| Cis (µM) LB-1 (µM) | 50 | 20 | 4 | 0.8 | 0.16 | 0 |
|---|---|---|---|---|---|---|
| 30 | 89.95 | 79.54 | 70.59 | 68.13 | 67.85 | 66.79 |
| 6 | 77.82 | 58.02 | 41.68 | 38.77 | 40.57 | 42.34 |
| 1.2 | 72.06 | 35.36 | 19.11 | 17.33 | 13.65 | 16.43 |
| 0.24 | 70.25 | 24.72 | 3.03 | -0.39 | -6.90 | -7.15 |
| 0.048 | 69.93 | 28.25 | 2.86 | -5.95 | -4.16 | -4.42 |
| 0 | 71.26 | 36.70 | 12.77 | 10.08 | 5.63 | 15.39 |
| monochlorinated myricetin (formula II)+cisplatin-STEDV | | | | | | |
| Cis (µM) LB-1 (µM) | 50 | 20 | 4 | 0.8 | 0.16 | 0 |
| 30 | 1.51 | 0.94 | 4.57 | 6.35 | 5.07 | 9.77 |
| 6 | 0.74 | 3.86 | 8.54 | 4.72 | 6.33 | 6.00 |
| 1.2 | 0.97 | 4.58 | 6.45 | 1.71 | 9.23 | 8.62 |
| 0.24 | 3.57 | 3.70 | 3.32 | 5.39 | 5.93 | 14.97 |
| 0.048 | 4.21 | 6.54 | 3.04 | 1.19 | 4.52 | 8.35 |
| 0 | 4.28 | 3.33 | 8.50 | 3.72 | 6.46 | 9.20 |

The highest inhibition rate is 89.95%, at concentration of 50 µM LB-1 and 30 µM cisplatin.
The inhibition rate is 71.26% at 50 µM LB-1 only, and the inhibition rate is 66.79% at 30 µM cisplatin only.

TABLE 74

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 72.31 | 68.09 | 66.32 | 66.33 | 64.26 | 64.00 |
| 200 | 70.38 | 65.63 | 62.89 | 61.89 | 64.57 | 63.24 |
| 40 | 68.48 | 65.21 | 63.86 | 63.72 | 63.38 | 62.89 |
| 8 | 63.26 | 53.11 | 54.06 | 54.22 | 52.66 | 51.97 |
| 1.6 | 34.55 | 18.01 | 14.74 | 12.54 | 15.25 | 13.95 |
| 0 | 30.66 | 9.11 | 7.41 | 3.71 | -0.66 | 2.59 |
| myricetin+taxol-STEDV | | | | | | |
| Tax (nM) Myr (µM) | 100 | 20 | 4 | 0.8 | 0.16 | 0 |
| 1000 | 1.55 | 2.80 | 2.44 | 3.05 | 2.78 | 3.53 |
| 200 | 1.07 | 1.99 | 2.52 | 2.12 | 1.96 | 0.45 |
| 40 | 0.60 | 1.03 | 1.20 | 1.90 | 1.83 | 2.20 |
| 8 | 1.82 | 3.29 | 3.09 | 2.54 | 4.46 | 4.37 |
| 1.6 | 1.79 | 4.37 | 1.35 | 3.58 | 5.52 | 6.86 |
| 0 | 4.32 | 5.85 | 4.46 | 3.18 | 11.69 | 5.03 |

The highest inhibitory rate is 72.31%, at concentration of 100uM myricetin and 1000 nM taxol.
The inhibition rate is 30.66% at 100 µM myricetin only, and the inhibition rate is 64% at 1000 nM taxol only.

TABLE 75

myricetin+doxorubicin
myricetin+doxorubicin-Relatively Cell inhibition of vehicle control(%)

| Dox (nM) Myr (pM) | 100 | 20 | 4 | 0.8 | 0.16 | 0 |
|---|---|---|---|---|---|---|
| 10000 | 83.76 | 78.95 | 76.42 | 75.29 | 75.37 | 76.46 |
| 2000 | 78.53 | 87.06 | 87.27 | 87.14 | 86.76 | 86.15 |
| 400 | 57.69 | 60.91 | 64.25 | 64.45 | 64.89 | 66.58 |
| 80 | 38.40 | 33.54 | 35.59 | 36.39 | 35.53 | 36.03 |
| 16 | 30.02 | 15.84 | 13.63 | 17.53 | 12.37 | 14.70 |
| 0 | 30.94 | 11.98 | 6.24 | 7.91 | 6.94 | 7.37 |

myricetin+doxorubicin-STEDV

| Dox (nM) Myr (pM) | 100 | 20 | 4 | 0.8 | 0.16 | 0 |
|---|---|---|---|---|---|---|
| 10000 | 1.36 | 0.66 | 1.43 | 3.11 | 3.00 | 2.51 |
| 2000 | 2.16 | 0.85 | 0.47 | 0.74 | 1.42 | 0.88 |
| 400 | 2.54 | 2.24 | 2.47 | 3.80 | 1.31 | 2.77 |
| 80 | 1.73 | 4.21 | 6.31 | 2.13 | 5.89 | 5.87 |
| 16 | 2.20 | 1.32 | 4.56 | 3.19 | 3.58 | 2.44 |
| 0 | 6.45 | 7.42 | 6.08 | 0.99 | 4.76 | 2.45 |

The highest inhibitory rate is 87.27%, at concentration of 4 µM myricetin and 2000 nM doxorubicin.
The inhibition rate is 6.24% at 4 µM myricetin only, and the inhibition rate is 86.15% at 2000 nM doxorubicin only.

TABLE 76

myricetin+topotecan
myricetin+topotecan-Relatively Cell inhibition of vehicle control(%)

| Top (nM) Myr (pM) | 100 | 20 | 4 | 0.8 | 0.16 | 0 |
|---|---|---|---|---|---|---|
| 10000 | 85.78 | 82.62 | 81.40 | 80.34 | 80.55 | 81.26 |
| 2000 | 92.11 | 90.64 | 89.53 | 88.88 | 88.67 | 88.92 |
| 400 | 79.89 | 74.94 | 73.77 | 74.60 | 72.96 | 74.56 |
| 80 | 64.59 | 54.44 | 51.56 | 52.05 | 52.51 | 54.35 |
| 16 | 32.89 | 17.45 | 14.23 | 10.63 | 7.86 | 13.17 |
| 0 | 23.34 | 5.10 | 0.67 | -6.16 | -4.32 | -6.38 |
| myricetin+topotecan-STEDV | | | | | | |
| Top (nM) Myr (pM) | 100 | 20 | 4 | 0.8 | 0.16 | 0 |
| 10000 | 0.83 | 1.27 | 0.50 | 0.77 | 0.26 | 0.28 |
| 2000 | 0.41 | 0.81 | 0.36 | 1.12 | 0.47 | 0.51 |
| 400 | 1.48 | 2.08 | 0.61 | 0.55 | 0.72 | 1.11 |
| 80 | 2.58 | 2.96 | 5.01 | 1.25 | 1.92 | 2.22 |
| 16 | 0.35 | 3.32 | 2.47 | 1.84 | 0.71 | 1.78 |
| 0 | 0.38 | 5.38 | 3.89 | 2.67 | 7.34 | 3.20 |

The highest inhibitory rate is 92.11%, at concentration of 100uM myricetin and 2000 nM topotecan.
The inhibition rate is 23.34% at 100 µM myricetin only, and the inhibition rate is 88.92% at 2000 nM topotecan only.

TABLE 77

myricetin+cisplatin
myricetin+cisolatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 86.28 | 82.68 | 81.83 | 79.84 | 79.17 | 79.85 |
| 6 | 60.24 | 51.79 | 52.36 | 48.99 | 48.95 | 49.43 |
| 1.2 | 43.76 | 23.76 | 23.31 | 24.28 | 22.24 | 19.41 |
| 0.24 | 29.16 | 9.83 | 4.76 | 1.41 | -3.49 | 1.60 |
| 0.048 | 22.29 | 1.80 | -1.49 | -3.40 | -11.64 | -11.79 |
| 0 | 31.40 | 10.48 | 6.73 | 1.28 | -5.76 | -1.59 |
| myricetin+cisplatin-STEDV | | | | | | |
| Cis (µM) Myr (µM) | 100 | 20 | 4 | 0.8 | 0.16 | 0 |
| 30 | 0.33 | 1.54 | 1.05 | 0.97 | 1.36 | 0.45 |
| 6 | 3.75 | 2.92 | 2.97 | 2.00 | 2.44 | 3.24 |
| 1.2 | 3.85 | 7.84 | 2.81 | 4.94 | 2.86 | 1.40 |
| 0.24 | 3.85 | 5.75 | 7.85 | 6.65 | 7.12 | 5.77 |
| 0.048 | 3.93 | 3.04 | 4.46 | 2.59 | 7.41 | 6.97 |
| 0 | 0.91 | 2.86 | 3.74 | 3.72 | 5.03 | 9.53 |

The highest inhibition rate is 86.28%, at concentration of 100 µM myricetin and 30 µM cisplatin.
The inhibition rate is 31.4% at 100 µM myricetin only, and the inhibition rate is 79.85% at 30 µM cisplatin only.

SK-OV-3

TABLE 78

monochlorinated myricetin (formula II)+taxol
monochlorinated myricetin (formula II)+taxol-Relatively Cell inhibition of vehicle control(%)

| Tax (nM) LB-1 (µM) | 50 | 20 | 4 | 0.8 | 0.16 | 0 |
|---|---|---|---|---|---|---|
| 1000 | 88.31 | 71.90 | 73.23 | 75.95 | 76.70 | 76.39 |
| 200 | 87.71 | 71.79 | 68.71 | 68.11 | 69.41 | 71.38 |
| 40 | 88.05 | 71.01 | 50.90 | 50.07 | 52.67 | 54.31 |
| 8 | 85.81 | 66.23 | 38.63 | 37.30 | 39.65 | 41.59 |
| 1.6 | 86.65 | 68.71 | 20.14 | 18.47 | 20.12 | 17.37 |
| 0 | 86.31 | 68.71 | 13.13 | 1.98 | 0.45 | 0.99 |
| monochlorinated myricetin (formula II)+taxol-STEDV | | | | | | |
| Tax (nM) LB-1 (µM) | 50 | 20 | 4 | 0.8 | 0.16 | 0 |
| 1000 | 0.24 | 0.78 | 1.23 | 0.98 | 0.26 | 0.54 |
| 200 | 0.76 | 3.79 | 0.50 | 1.30 | 0.29 | 0.76 |
| 40 | 0.93 | 1.93 | 1.54 | 0.97 | 0.74 | 1.78 |
| 8 | 0.13 | 1.16 | 0.65 | 1.54 | 1.74 | 0.89 |
| 1.6 | 0.80 | 1.72 | 2.41 | 3.79 | 1.43 | 9.87 |
| 0 | 0.57 | 0.84 | 1.48 | 2.32 | 4.10 | 5.20 |

The highest inhibition rate is 88.31%, at concentration of 50uM LB-1 and 1000 nM taxol.
The inhibition rate is 86.31% at 50 µM LB-1 only, and the inhibition rate is 76.39% at 1000 nM taxol only.

TABLE 79

monochlorinated myricetin (formula II)+doxorubicin
monochlorinated myricetin (formula II)+doxorubicin-Relatively Cell inhibition of vehicle control(%)

| Dox (nM) LB-1 (µM) | 50 | 20 | 4 | 0.8 | 0.16 | 0 |
|---|---|---|---|---|---|---|
| 10000 | 85.69 | 85.27 | 85.63 | 83.52 | 84.14 | 84.35 |
| 2000 | 95.51 | 92.31 | 91.92 | 90.78 | 91.38 | 91.55 |
| 400 | 92.85 | 83.22 | 73.16 | 67.22 | 70.41 | 72.01 |
| 80 | 87.84 | 68.55 | 43.88 | 32.38 | 35.85 | 40.49 |
| 16 | 84.77 | 65.82 | 8.42 | -9.10 | -0.40 | 2.75 |
| 0 | 85.34 | 65.41 | 11.21 | -11.14 | -1.54 | -0.33 |

monochlorinated myricetin (formula II)+doxorubicin-STEDV

| | | | | | | |
|---|---|---|---|---|---|---|
| Dox (nM) LB-1 (µM) | 50 | 20 | 4 | 0.8 | 0.16 | 0 |
| 10000 | 0.23 | 3.34 | 0.52 | 0.40 | 2.31 | 0.96 |
| 2000 | 0.23 | 0.87 | 0.54 | 0.71 | 0.26 | 0.30 |
| 400 | 1.29 | 2.45 | 0.64 | 2.11 | 1.34 | 1.27 |
| 80 | 0.94 | 1.68 | 2.17 | 1.11 | 2.87 | 2.54 |
| 16 | 1.63 | 4.57 | 1.70 | 2.46 | 9.15 | 3.01 |
| 0 | 2.36 | 2.57 | 2.65 | 6.82 | 6.80 | 4.77 |

The highest inhibition rate is 95.51%, at concentration of 50 µM LB-1 and 2000 nM doxorubicin.
The inhibition rate is 85.34% at 50 µM LB-1 only, and the inhibition rate is 91.55% at 2000 nM doxorubicin only.

TABLE 80

monochlorinated myricetin (formula II)+topotecan
monochlorinated myricetin (formula II)+topotecan-Relatively Cell inhibition of vehicle control(%)

| Top (nM) LB-1 (µM) | 50 | 20 | 4 | 0.8 | 0.16 | 0 |
|---|---|---|---|---|---|---|
| 10000 | 96.23 | 95.62 | 93.89 | 93.73 | 94.04 | 93.89 |
| 2000 | 94.32 | 92.91 | 90.24 | 89.76 | 90.06 | 90.55 |
| 400 | 90.90 | 87.34 | 82.90 | 81.12 | 82.91 | 83.70 |
| 80 | 88.46 | 82.65 | 70.28 | 67.53 | 68.25 | 68.74 |
| 16 | 86.57 | 73.10 | 31.75 | 15.14 | 21.71 | 23.00 |
| 0 | 87.01 | 68.44 | 12.65 | -0.66 | 4.12 | 8.46 |
| monochlorinated myricetin (formula II)+topotecan-STEDV | | | | | | |
| Top (nM) LB-1 (µM) | 50 | 20 | 4 | 0.8 | 0.16 | 0 |
| 10000 | 0.35 | 0.13 | 0.37 | 0.47 | 0.80 | 0.43 |
| 2000 | 0.82 | 0.65 | 0.42 | 0.75 | 0.73 | 0.14 |
| 400 | 0.41 | 0.70 | 0.14 | 1.02 | 1.16 | 1.07 |
| 80 | 0.97 | 1.24 | 0.96 | 1.96 | 2.85 | 2.82 |
| 16 | 0.90 | 1.93 | 3.33 | 4.05 | 4.38 | 1.37 |
| 0 | 0.37 | 1.15 | 4.79 | 5.87 | 4.47 | 3.27 |

The highest inhibition rate is 96.23%, at concentration of 50uM LB-1 and 10000 nM topotecan.
The inhibition rate is 87.01% at 50 µM LB-1 only, and the inhibition rate is 93.89% at 1000 nM topotecan only.

TABLE 81

monochlorinated myricetin formula II)+cisplatin
monochlorinated myricetin (formula II)+cisplatin-Relatively Cell inhibition of vehicle control(%)

| Cis (µM) B-1 (µM) | 50 | 20 | 4 | 0.8 | 0.16 | 0 |
|---|---|---|---|---|---|---|
| 30 | 90.83 | 93.31 | 89.34 | 89.18 | 88.94 | 88.98 |
| 6 | 91.44 | 77.48 | 53.86 | 49.66 | 48.23 | 50.05 |
| 1.2 | 86.67 | 70.23 | 14.37 | 3.23 | 4.49 | 7.66 |
| 0.24 | 85.31 | 66.82 | 2.33 | -5.53 | -5.01 | -1.86 |
| 0.048 | 85.51 | 67.51 | 4.02 | -3.30 | -3.17 | -0.61 |
| 0 | 87.21 | 68.34 | 9.67 | -0.42 | -0.42 | 5.22 |
| monochlorinated myricetin (formula II)+cisplantin-STEDV | | | | | | |
| Cis (µM) LB-1 (µM) | 50 | 20 | 4 | 0.8 | 0.16 | 0 |
| 30 | 0.38 | 0.41 | 0.51 | 0.32 | 0.47 | 0.47 |
| 6 | 0.61 | 0.62 | 2.87 | 3.89 | 3.67 | 2.76 |
| 1.2 | 0.77 | 1.18 | 2.47 | 1.72 | 2.54 | 1.96 |
| 0.24 | 0.19 | 0.25 | 1.73 | 4.70 | 0.66 | 2.73 |
| 0.048 | 0.84 | 2.19 | 1.77 | 1.02 | 1.76 | 0.40 |
| 0 | 0.81 | 0.12 | 1.01 | 3.05 | 0.39 | 1.81 |

The highest inhibition rate is 93.31%, at concentration of 20 µM LB-1 and 30µMcisplatin.
The inhibition rate is 68.34% at 50 µM LB-1 only, and the inhibition rate is 88.98% at 30 µM cisplatin only.

TABLE 82

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 92.69 | 75.99 | 72.22 | 72.62 | 72.82 | 73.38 |
| 200 | 91.51 | 73.29 | 68.48 | 67.56 | 67.64 | 68.68 |
| 40 | 90.14 | 55.84 | 50.91 | 49.81 | 49.88 | 52.55 |
| 8 | 89.06 | 45.94 | 39.86 | 40.63 | 40.88 | 39.88 |
| 1.6 | 88.63 | 18.81 | 16.94 | 15.83 | 17.84 | 18.71 |
| 0 | 87.91 | 7.54 | 0.87 | 0.71 | 2.23 | 2.11 |
| myricetin+taxol-STEDV | | | | | | |
| Tax (nM) Myr (µM) | 100 | 20 | 4 | 0.8 | 0.16 | 0 |
| 1000 | 0.50 | 1.09 | 0.56 | 0.67 | 1.81 | 0.67 |
| 200 | 0.79 | 0.84 | 0.66 | 0.70 | 1.36 | 1.66 |
| 40 | 1.05 | 3.01 | 1.29 | 0.69 | 0.73 | 1.64 |
| 8 | 0.34 | 2.49 | 0.33 | 0.92 | 0.69 | 2.06 |
| 1.6 | 3.13 | 3.24 | 1.95 | 1.52 | 1.62 | 0.37 |
| 0 | 3.82 | 1.23 | 3.04 | 1.39 | 3.67 | 2.89 |

The highest inhibitory rate is 92.69%, at concentration of 100uM myricetin and 1000 nM taxol.
The inhibition rate is 87.91% at 100 µM myricetin only, and the inhibition rate is 73.38% at 1000 nM taxol only.

TABLE 83

myricetin+doxorubicin
myricetin+doxorubicin-Relatively Cell inhibition of vehicle control(%)

| Dox (nM) yr (µM) | 100 | 20 | 4 | 0.8 | 0.16 | 0 |
|---|---|---|---|---|---|---|
| 10000 | 98.68 | 89.82 | 86.81 | 85.73 | 85.43 | 85.70 |
| 2000 | 93.99 | 88.36 | 91.64 | 91.03 | 91.17 | 91.18 |
| 400 | 90.30 | 67.02 | 69.60 | 70.36 | 68.84 | 69.42 |
| 80 | 86.55 | 25.39 | 33.96 | 35.62 | 33.78 | 36.36 |
| 16 | 88.85 | 5.35 | 3.88 | -3.36 | -3.56 | 1.98 |
| 0 | 91.33 | 1.89 | 1.70 | 0.36 | 3.45 | 3.71 |
| myricetin+doxorubicin-STEDV | | | | | | |
| Dox (nM) Myr (µM) | 100 | 20 | 4 | 0.8 | 0.16 | 0 |
| 10000 | 1.22 | 1.84 | 0.96 | 0.80 | 1.33 | 0.97 |
| 2000 | 1.17 | 1.34 | 0.58 | 0.52 | 0.30 | 0.14 |
| 400 | 1.98 | 2.72 | 1.70 | 1.53 | 1.83 | 1.13 |
| 80 | 5.24 | 3.07 | 3.81 | 3.30 | 1.79 | 4.95 |
| 16 | 1.67 | 4.99 | 1.86 | 3.35 | 2.74 | 0.85 |
| 0 | 3.04 | 4.35 | 4.52 | 4.71 | 1.23 | 4.09 |

The highest inhibitory rate is 98.68%, at concentration of 100 µM myricetin and 10000 nM doxorubicin.
The inhibition rate is 91.33% at 100 µM myricetin only, and the inhibition rate is 85.7% at 10000 nM doxorubicin only.

TABLE 84

myricetin+topotecan
myricetin+topotecan-Relatively Cell inhibition of vehicle control(%)

| TOP (nM) Myr (µM) | 100 | 20 | 4 | 0.8 | 0.16 | 0 |
|---|---|---|---|---|---|---|
| 10000 | 96.35 | 95.58 | 94.94 | 94.47 | 94.18 | 94.18 |
| 2000 | 93.64 | 92.11 | 91.36 | 90.62 | 90.14 | 90.97 |
| 400 | 91.77 | 84.19 | 83.36 | 81.86 | 82.57 | 82.50 |
| 80 | 91.62 | 72.64 | 68.71 | 67.11 | 67.92 | 67.77 |
| 16 | 89.98 | 23.67 | 22.14 | 15.16 | 17.17 | 21.67 |
| 0 | 87.18 | 0.29 | 3.31 | 4.27 | 2.30 | 7.11 |
| myricetin+topotecan-STEDV | | | | | | |
| Top (nM) Myr (µM) | 100 | 20 | 4 | 0.8 | 0.16 | 0 |
| 10000 | 0.70 | 0.15 | 0.18 | 0.33 | 0.15 | 0.64 |
| 2000 | 0.05 | 0.12 | 0.37 | 0.29 | 0.72 | 0.42 |
| 400 | 1.34 | 0.29 | 0.65 | 0.64 | 1.42 | 0.04 |
| 80 | 0.74 | 0.27 | 2.64 | 1.01 | 1.15 | 2.32 |
| 16 | 1.38 | 6.16 | 4.46 | 3.05 | 3.73 | 2.33 |
| 0 | 1.30 | 1.60 | 8.09 | 4.74 | 4.91 | 1.53 |

The highest inhibitory rate is 96.35%, at concentration of 100uM myricetin and 10000 nM topotecan.
The inhibition rate is 87.18% at 100 µM myricetin only, and the inhibition rate is 94.18% at 10000 nM topotecan only.

TABLE 85

myricetin+cisplatin
myricetin+cilatin-Relatively Cell inhibition of vehicle control(%)

| Cis (µM)Myr (µM) | 100 | 20 | 4 | 0.8 | 0.16 | 0 |
|---|---|---|---|---|---|---|
| 30 | 97.69 | 85.73 | 84.83 | 84.82 | 85.22 | 85.03 |
| 6 | 96.17 | 62.79 | 57.74 | 56.51 | 56.69 | 55.97 |
| 1.2 | 89.87 | 28.03 | 18.79 | 18.21 | 18.35 | 17.32 |
| 0.24 | 90.29 | 5.11 | -1.58 | -5.17 | -2.83 | -1.44 |
| 0.048 | 91.25 | 6.25 | -1.12 | -2.72 | -2.75 | 0.65 |
| 0 | 89.47 | 9.41 | 2.94 | 2.08 | 1.84 | 0.33 |
| myricetin+cisplatin-STEDV | | | | | | |
| Cis (µM)Myr (µM) | 100 | 20 | 4 | 0.8 | 0.16 | 0 |
| 30 | 1.51 | 0.10 | 0.67 | 0.45 | 0.16 | 0.40 |
| 6 | 0.38 | 0.72 | 1.14 | 1.15 | 3.03 | 1.30 |
| 1.2 | 2.18 | 1.97 | 0.88 | 1.26 | 1.56 | 1.76 |
| 0.24 | 0.38 | 2.48 | 3.83 | 0.31 | 4.75 | 0.80 |
| 0.048 | 0.39 | 3.36 | 2.17 | 1.45 | 2.06 | 2.34 |
| 0 | 1.43 | 2.92 | 3.48 | 1.08 | 1.84 | 2.12 |

The highest inhibition rate is 97.69%, at concentration of 100 µM myricetin and 30 µM cisplatin.
The inhibition rate is 89.47% at 100 µM myricetin only, and the inhibition rate is 85.03% at 30 µM cisplatin only.

A549

TABLE 86

monochlorinated myricetin (formula II)+taxol
monochlorinated myricetin (formula II)+taxol-Relatively Cell inhibition of vehicle control(%)

| Tax (nM) LB-1 (µM) | 50 | 20 | 4 | 0.8 | 0.16 | 0 |
|---|---|---|---|---|---|---|
| 1000 | 80.24 | 77.84 | 77.18 | 77.21 | 76.30 | 76.64 |
| 200 | 80.95 | 78.94 | 77.03 | 78.05 | 76.93 | 77.44 |
| 40 | 80.33 | 72.80 | 70.76 | 71.00 | 69.30 | 68.79 |
| 8 | 79.66 | 58.53 | 56.16 | 55.21 | 55.64 | 55.11 |
| 1.6 | 77.87 | 21.85 | 17.04 | 17.36 | 16.92 | 13.66 |
| 0 | 75.25 | 6.46 | -0.94 | 0.09 | 1.27 | 1.37 |
| monochlorinated myricetin (formula II)+taxol-STEDV | | | | | | |
| Tax (nM) LB-1 (µM) | 50 | 20 | 4 | 0.8 | 0.16 | 0 |
| 1000 | 0.37 | 0.78 | 0.82 | 0.75 | 0.24 | 1.04 |
| 200 | 0.74 | 1.56 | 0.83 | 0.47 | 0.24 | 1.11 |
| 40 | 0.72 | 0.66 | 0.56 | 1.27 | 1.21 | 0.56 |
| 8 | 0.64 | 0.36 | 1.60 | 0.49 | 0.41 | 2.56 |
| 1.6 | 0.62 | 0.95 | 1.54 | 1.57 | 1.01 | 0.65 |
| 0 | 1.54 | 1.72 | 2.53 | 1.76 | 1.73 | 1.89 |

The highest inhibition rate is 80.95%, at concentration of 50uM LB-1 and 200 nM taxol.
The inhibition rate is 75.25% at 50 µM LB-1 only, and the inhibition rate is 77.44% at 200 nM taxol only.

TABLE 87

monochlorinated myricetin formula ll+doxorubicin
monochlorinated myricetin (formula II)+doxorubicin-Relatively Cell inhibition of vehicle control(%)

| Dox (nM) LB-1 (pM) | 50 | 20 | 4 | 0.8 | 0.16 | 0 |
|---|---|---|---|---|---|---|
| 10000 | 93.69 | 94.41 | 94.18 | 93.91 | 94.31 | 94.29 |
| 2000 | 96.18 | 97.56 | 97.22 | 96.56 | 96.33 | 95.63 |
| 400 | 82.95 | 62.55 | 61.53 | 62.50 | 62.64 | 61.50 |
| 80 | 76.15 | 46.48 | 44.84 | 46.59 | 45.12 | 45.26 |
| 16 | 75.19 | 14.63 | 20.36 | 18.90 | 19.66 | 17.90 |
| 0 | 73.75 | 5.53 | -1.25 | -2.38 | -0.28 | -0.30 |
| monochlorinated myricetin (formula II)+doxorubicin-STEDV | | | | | | |
| Dox (nM) LB-1 (pM) | 50 | 20 | 4 | 0.8 | 0.16 | 0 |
| 10000 | 0.69 | 0.44 | 0.45 | 0.51 | 0.69 | 0.47 |
| 2000 | 0.24 | 0.31 | 0.21 | 0.28 | 0.40 | 0.41 |
| 400 | 0.76 | 1.80 | 1.75 | 0.86 | 2.13 | 2.38 |
| 80 | 1.56 | 1.44 | 2.37 | 0.87 | 2.10 | 2.54 |
| 16 | 0.75 | 2.63 | 2.22 | 5.69 | 0.53 | 2.69 |
| 0 | 1.11 | 1.78 | 1.33 | 1.98 | 2.46 | 3.04 |

The highest inhibition rate is 97.56%, at concentration of 20 µM LB-1 and 2000 nM doxorubicin.
The inhibition rate is 5.53% at 20 µM LB-1 only, and the inhibition rate is 95.63% at 2000 nM doxorubicin only.

TABLE 88

monochlorinated myricetin formula II)+topotecan
monochlorinated myricetin (formula II)+topotecan-Relatively Cell inhibition of vehicle control(%)

| Top (nM) LB-1(pM) | 50 | 20 | 4 | 0.8 | 0.16 | 0 |
|---|---|---|---|---|---|---|
| 10000 | 99.39 | 95.45 | 94.91 | 94.97 | 94.5 | 93.99 |
| 2000 | 97.09 | 82.35 | 81.81 | 80.56 | 81.57 | 80.57 |
| 400 | 90.25 | 64.92 | 64.90 | 62.71 | 62.88 | 62.21 |
| 80 | 81.68 | 37.68 | 33.60 | 30.64 | 30.64 | 37.94 |
| 16 | 76.39 | 9.76 | 2.24 | -0.58 | -0.50 | -0.68 |
| 0 | 75.89 | 9.03 | 2.04 | 1.13 | 0.49 | 0.36 |

monochlorinated myricetin formula II)+topotecan-STEDV

| Top (nM) LB-1(pM) | 50 | 20 | 4 | 0.8 | 0.16 | 0 |
|---|---|---|---|---|---|---|
| 10000 | 0.07 | 0.11 | 0.76 | 0.47 | 0.37 | 0.14 |
| 2000 | 0.17 | 1.08 | 0.84 | 1.04 | 1.00 | 0.51 |
| 400 | 0.63 | 1.80 | 0.81 | 2.55 | 0.93 | 0.92 |
| 80 | 0.53 | 3.71 | 2.28 | 3.26 | 2.34 | 0.28 |
| 16 | 0.48 | 1.56 | 2.75 | 2.43 | 0.97 | 1.23 |
| 0 | 1.14 | 3.03 | 3.80 | 3.28 | 2.01 | 2.03 |

The highest inhibition rate is 99.39%, at concentration of 50uM LB-1 and 10000 nM topotecan.
The inhibition rate is 75.89% at 50 µM LB-1 only, and the inhibition rate is 93.99% at 10000 nM cisplatin only.

TABLE 89

monochlorinated myricetin formula II+cisplatin
monochlorinated myricetin (formula II)+cisplatin-Relatively Cell inhibition of vehicle control(%)

| Cis (µM) LB-1 (µM) | 50 | 20 | 4 | 0.8 | 0.16 | 0 |
|---|---|---|---|---|---|---|
| 30 | 95.00 | 65.48 | 63.72 | 62.92 | 62.30 | 65.03 |
| 6 | 78.88 | 34.70 | 32.10 | 30.61 | 32.99 | 33.27 |
| 1.2 | 74.86 | 5.98 | 1.88 | -4.86 | 1.80 | 1.57 |
| 0.24 | 74.31 | 9.00 | 4.83 | 1.16 | 0.91 | 0.66 |
| 0.048 | 72.63 | 8.89 | 0.67 | -1.01 | -0.38 | 0.17 |
| 0 | 74.82 | 9.07 | 1.57 | 0.59 | 2.19 | -0.51 |
| monochlorinated myricetin (formula II)+cisplatin-STEDV | | | | | | |
| Cis (µM) LB-1 (µM) | 50 | 20 | 4 | 0.8 | 0.16 | 0 |
| 30 | 0.57 | 3.67 | 2.85 | 3.24 | 4.18 | 2.01 |
| 6 | 2.55 | 4.00 | 3.62 | 1.28 | 1.38 | 0.47 |
| 1.2 | 0.58 | 5.33 | 1.03 | 5.00 | 2.96 | 1.16 |
| 0.24 | 0.87 | 0.96 | 1.05 | 0.90 | 2.02 | 1.97 |
| 0.048 | 0.85 | 2.95 | 2.40 | 3.21 | 2.02 | 0.53 |
| 0 | 1.06 | 1.64 | 2.21 | 1.52 | 1.27 | 0.85 |

The highest inhibition rate is 95.00%, at concentration of 50 µM LB-1 and 30 µM cisplatin.
The inhibition rate is 74.82% at 50 µM LB-1 only, and the inhibition rate is 65.03% at 30 µM cisplatin only.

### Myricetin Taxol

The inhibition rate is 74.82% at 50 µM LB-1 only, and the inhibition rate is 65.03% at 30 µM cisplatin only.

TABLE 90

myricetin+taxol
myricetin+taxol-Relatively Cell inhibition of vehicle control(%)

| Tax (nM) Myr (µM) | 100 | 20 | 4 | 0.8 | 0.16 | 0 |
|---|---|---|---|---|---|---|
| 1000 | 78.00 | 79.36 | 77.86 | 76.89 | 76.95 | 77.27 |
| 200 | 78.20 | 78.59 | 78.23 | 76.33 | 76.61 | 75.24 |
| 40 | 73.71 | 71.66 | 69.29 | 68.78 | 69.48 | 68.03 |
| 8 | 66.97 | 57.45 | 55.28 | 54.20 | 54.45 | 53.76 |
| 1.6 | 50.61 | 16.97 | 11.41 | 7.31 | 10.20 | 7.89 |
| 0 | 41.11 | 9.01 | 3.22 | -0.98 | 1.81 | -1.35 |
| myricetin+taxol-STEDV | | | | | | |
| Tax (nM) Myr (µM) | 100 | 20 | 4 | 0.8 | 0.16 | 0 |
| 1000 | 1.40 | 1.62 | 1.13 | 1.49 | 1.03 | 0.45 |
| 200 | 0.95 | 1.48 | 0.50 | 1.50 | 1.22 | 1.28 |
| 40 | 1.96 | 1.73 | 1.72 | 2.44 | 1.96 | 2.39 |
| 8 | 1.18 | 2.05 | 1.52 | 2.56 | 2.89 | 1.14 |
| 1.6 | 2.15 | 2.04 | 2.16 | 1.62 | 3.17 | 2.09 |
| 0 | 4.24 | 0.41 | 1.16 | 3.71 | 1.32 | 2.01 |

The highest inhibitory rate is 79.36%, at concentration of 20uM myricetin and 1000 nM taxol.
The inhibition rate is 9.01% at 20 µM myricetin only, and the inhibition rate is 77.27% at 1000 nM taxol only.

TABLE 91

myricetin+doxorubicin
myricetin+doxorubicin-Relatively Cell inhibition of vehicle control(%)

| Dox (nM) Myr (µM) | 100 | 20 | 4 | 0.8 | 0.16 | 0 |
|---|---|---|---|---|---|---|
| 10000 | 96.94 | 95.36 | 95.08 | 94.57 | 94.72 | 94.26 |
| 2000 | 81.41 | 92.64 | 95.62 | 95.87 | 96.36 | 95.76 |
| 400 | 66.37 | 60.66 | 62.49 | 63.05 | 63.51 | 61.43 |
| 80 | 53.79 | 43.41 | 44.03 | 45.39 | 46.29 | 42.17 |
| 16 | 44.23 | 9.57 | 13.46 | 12.52 | 17.63 | 14.74 |
| 0 | 39.40 | 7.98 | 2.41 | -2.56 | 2.87 | -1.22 |
| myricetin+doxorubicin-STEDV | | | | | | |
| Dox (nM) Myr (µM) | 100 | 20 | 4 | 0.8 | 0.16 | 0 |
| 10000 | 0.22 | 0.57 | 0.23 | 0.62 | 0.23 | 0.17 |
| 2000 | 3.28 | 1.78 | 0.62 | 0.84 | 0.63 | 0.67 |
| 400 | 2.32 | 3.52 | 3.04 | 2.35 | 2.16 | 1.72 |
| 80 | 3.20 | 3.00 | 1.47 | 2.82 | 0.87 | 2.74 |
| 16 | 2.21 | 1.86 | 2.21 | 4.74 | 0.23 | 3.76 |
| 0 | 2.87 | 2.32 | 1.68 | 1.20 | 1.75 | 0.76 |

The highest inhibitory rate is 96.94%, at concentration of 100 µM myricetin and 1000 nM doxorubicin.
The inhibition rate is 39.4% at 100 µM myricetin only, and the inhibition rate is 94.26% at 10000 nM doxorubicin only.

TABLE 92

myricetin+topotecan
myricetin+topotecan-Relatively Cell inhibition of vehicle control(%)

| Top (nM) Myr (µM) | 100 | 20 | 4 | 0.8 | 0.16 | 0 |
|---|---|---|---|---|---|---|
| 10000 | 96.32 | 95.05 | 94.71 | 94.55 | 94.19 | 94.37 |
| 2000 | 86.55 | 82.04 | 81.06 | 80.81 | 81.15 | 79.74 |
| 400 | 75.14 | 63.94 | 61.87 | 62.03 | 63.01 | 59.95 |
| 80 | 61.69 | 36.52 | 29.25 | 26.32 | 27.64 | 34.87 |
| 16 | 46.15 | 10.11 | 3.84 | 0.70 | 1.49 | 2.50 |
| 0 | 39.89 | 9.45 | 2.40 | -2.74 | 0.33 | -1.60 |

myricetin+topotecan-STEDV

| Top (nM) Myr (µM) | 100 | 20 | 4 | 0.8 | 0.16 | 0 |
|---|---|---|---|---|---|---|
| 10000 | 0.47 | 0.36 | 0.22 | 0.50 | 0.78 | 0.20 |
| 2000 | 0.35 | 1.47 | 0.22 | 0.74 | 0.73 | 0.49 |
| 400 | 1.74 | 0.62 | 0.99 | 2.07 | 0.53 | 1.54 |
| 80 | 1.24 | 1.92 | 0.98 | 2.46 | 0.46 | 2.64 |
| 16 | 2.16 | 2.22 | 2.00 | 2.44 | 1.32 | 3.13 |
| 0 | 1.90 | 2.48 | 0.90 | 2.30 | 2.27 | 1.78 |

The highest inhibitory rate is 96.32%, at concentration of 100uM myricetin and 10000 nM topotecan.
The inhibition rate is 39.89% at 100 µM myricetin only, and the inhibition rate is 94.37% at 10000 nM topotecan only.

TABLE 93

myricetin+cisplatin
myricetin+cisplatin-Relatively Cell inhibition of vehicle control(%)

| Cis (µM) Myr (µM) | 100 | 20 | 4 | 0.8 | 0.16 | 0 |
|---|---|---|---|---|---|---|
| 30 | 89.71 | 80.50 | 79.45 | 81.83 | 78.47 | 77.27 |
| 6 | 61.32 | 39.51 | 34.21 | 31.25 | 32.18 | 33.19 |
| 1.2 | 45.34 | 8.94 | 3.64 | 1.80 | -0.63 | 0.61 |
| 0.24 | 41.28 | 7.10 | 1.83 | -1.47 | 0.43 | 0.04 |
| 0.048 | 40.35 | 9.32 | 0.99 | -1.90 | 2.03 | 1.46 |
| 0 | 40.33 | 7.42 | 0.85 | -3.15 | 1.12 | 0.47 |

myricetin+cisplatin-STEDV

| Cis (µM) Myr (µM) | 100 | 20 | 4 | 0.8 | 0.16 | 0 |
|---|---|---|---|---|---|---|
| 30 | 0.47 | 0.91 | 1.84 | 1.25 | 0.88 | 0.24 |
| 6 | 2.00 | 3.28 | 0.68 | 1.19 | 1.07 | 0.99 |
| 1.2 | 1.87 | 4.02 | 2.36 | 3.70 | 3.06 | 2.46 |
| 0.24 | 3.68 | 1.36 | 0.35 | 1.39 | 1.21 | 1.39 |
| 0.048 | 0.51 | 1.10 | 1.15 | 0.93 | 3.95 | 1.47 |
| 0 | 2.96 | 5.00 | 3.13 | 2.18 | 3.70 | 2.45 |

The highest inhibition rate is 89.71%, at concentration of 100 µM myricetin and 30 µM cisplatin.
The inhibition rate is 40.33% at 100 µM myricetin only, and the inhibition rate is 77.27% at 30 µM cisplatin only.

Although the invention has been described above in relation to preferred embodiments thereof, it will be understood by those skilled in the art that variations and modifications can be accomplished in these preferred embodiments without departing from the scope and spirit of the invention.

1. A pharmaceutical composition comprising:
myricetin; and
an anticancer chemotherapeutic agent.

2. The pharmaceutical composition of claim 1, wherein the anticancer chemotherapeutic agent is selected from the group consisting of paclitaxel, doxorubicin, topotecan and cisplatin.

3. The pharmaceutical composition of claim 1, wherein myricetin comprises myricetin or a halogenated form thereof.

4. The pharmaceutical composition of claim 3, wherein the halogenated myricetin comprised chlorinated myricetin.

5. The pharmaceutical composition of claim 4, wherein chlorinated myricetin is selected from the group consisting of at least one of monochlorinated myricetin and dichlorinated myricetin.

6. The pharmaceutical composition of claim 1, wherein the chlorinated myricetin has formula (I) 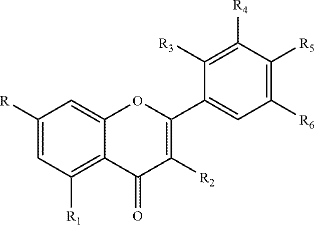
wherein:
R, R₁, R₂, R₄, R₅, and R₆ are a hydroxyl group or chlorine,
R₃ is hydrogen; and
at least one of R, R₁, R₂, R₄, R₅, and R₆ is chlorine.

7. The pharmaceutical composition of claim 3, wherein monochlorinated myricetin has chemical formula II: 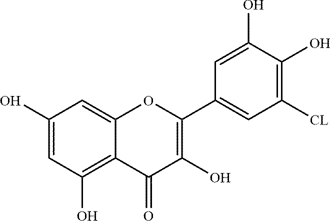
.

8. The pharmaceutical composition of claim 3, wherein dichlorinated myricetin has chemical formula III: 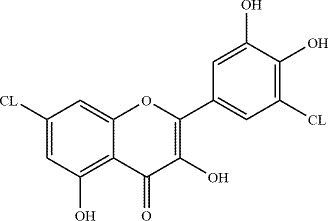
.

9. The pharmaceutical composition of claim 1, wherein the composition is effective to treat or limit the occurrence of cancers selected from the group consisting of breast cancer, glioblastoma, prostate adenocarcinoma, kidney cancer, gastric cancer, colorectal cancer, liver cancer, pancreatic cancer, ovarian cancer, and lung adenocarcinoma, when administered to a patient in need of treatment therefrom.

10. A method of treating or limiting the occurrence of cancer comprising co-administering a therapeutically effective amount of myricetin; and an anticancer chemotherapeutic agent, to a patient in need of treatment therefrom.

11. The method of claim 10, wherein the myricetin is selected from the group consisting of at least one of monochlorinated myricetin and dichlorinated myricetin.

12. The method of claim 10, wherein the chlorinated myricetin and the anticancer chemotherapeutic agent are formulated together in a pharmaceutical composition and the co-administering chlorinated myricetin and the chemotherapeutic agent comprises administering the pharmaceutical composition.

13. The method of claim 10, wherein the anticancer chemotherapeutic agent is selected from the group consisting of paclitaxel, doxorubicin, topotecan and cisplatin.

14. The method of claim 10, wherein the monochlorinated myricetin has chemical formula I: 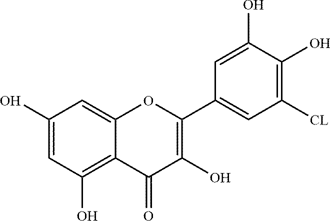
.

15. The method of claim 10, wherein dichlorinated myricetin has chemical formula II: 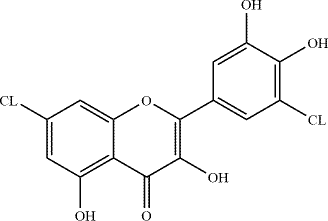
.

16. The method of claim 10, wherein the chlorinated myricetin has formula (I) 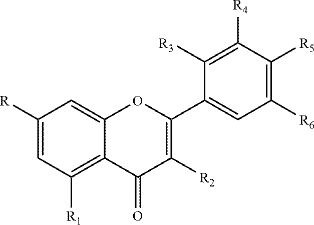
wherein:
R, R₁, R₂, R₄, R₅, and R₆ are a hydroxyl group or chlorine,
R₃ is hydrogen; and
at least one of R, R₁, R₂, R₄, R₅, and R₆ is chlorine.

17. The method of claim 10, wherein the cancer is selected from the group consisting of breast cancer, glioblastoma, prostate adenocarcinoma, kidney cancer, gastric cancer, colorectal cancer, liver cancer, pancreatic cancer, ovarian cancer, and lung adenocarcinoma.

18. A method of down regulating proviral integration site for a moloney murine leukemia virus (PIM) kinase, comprising co-administering a therapeutically effective amount of myricetin; and an anticancer chemotherapeutic agent, to a patient in need of treatment therefrom.

19. The method of claim 18, wherein the PIM kinase is selected from the group consisting of PIM-1, PIM-2 and PIM-3.

20. The method of claim 18, wherein downregulating PIM kinase results in the upregulation or stimulative expression tumor suppressor mechanisms including retinoblastoma protein (pRb), cyclin dependent kinase inhibitor 2A, CDKN2A multiple tumor suppressor 1 (p16), ARF tumor suppressor (p14arf), transforming growth factor (TGF-beta), adenomatous polyposis coli (APC), breast cancer type 1 susceptibility protein (BRCA1), and / or tumor protein / cellular tumor antigen (p53).

21. The method of claim 18, wherein downregulating PIM kinase results in the upregulation or simulative expression of metastasis suppressor proteins including breast cancer metastasis suppressor 1 (BRMS1), mediator of RNA polymerase II transcription subunit 23 (CRSP3), developmentally regulated GTP binding protein1 (DRG1), cluster/differentiation 82 (CD82), serum deprived response protein (SDPR), kisseptin 54 (KISS1), nucleoside diphosphate kinase A (NME1), tissue inhibitor of metalloproteinase (TIMPs), or dual specificity mitogen activated protein kinase 4 (MKK4).

22. The method of claim 18, wherein downregulating PIM kinase results in downrange attenuation of pro-inflammatory regulators including tumor necrosis factor alpha (TNF-α), nuclear factor kappa light chain enhancer of activated B cells (NF-kB), or lymphotoxin beta receptor (LTBR).

23. The method of claim 18, wherein downregulating PIM kinase results in inhibition of downrange pro-oncogenetic driving mechanisms and gene expression including such as chromobox protein homolog 3 (CBX3), m-phase inducer phosphatase 1 (CDC24A), heat shock protein 90kDa alpha-member A1, nuclear factor / activated T-cells-cytoplasmic 1 (NFATC1), nuclear mitotic apparatus protein 1, cyclin dependent kinase inhibitor 1 (P21), staphylococcal nuclease domain containing protein 1 (SND1), transcription factor p65 (RELA), dual-specificity phosphatase (Cdc25), P13K Kinase, hypoxia induced factor 1 alpha (HIF1A), MYC, Janus kinase signal transducer and activator of transcription proteins (JAK-STAT), mammalian target of rapamycin (mTOR), FK506 binding protein 12 rapamycin associated protein 1 (FRAP1), protein kinase B, basic fibroblast growth factor (FGF2), vascular endothelial growth factor (VEGF), proto-oncogene (RET), Ras GTPase, RAF proto-oncogene serine/therine protein kinase (c-Raf), cyclin dependent kinase (CDK), Tyrosine-protein kinase (SYK), platelet derived growth factor (PDGF).

24. The method of claim 18, wherein myricetin comprises myricetin or a halogenated form thereof.
